(19) United States
(12) Patent Application Publication
(10) Pub. No.: US 2024/0150747 A1
(43) Pub. Date: May 9, 2024

(54) ENCYSTMENT AND STABIILISATION OF CILIATE PROTOZOA CELLS

(71) Applicants: The University of Melbourne, Victoria (AU); Grains Research and Development Corporation, Barton (AU)

(72) Inventors: Ruth Elizabeth Haites, Victoria (AU); Helen Jacobe, Victoria (AU)

(21) Appl. No.: 18/281,486

(22) PCT Filed: Mar. 17, 2021
(86) PCT No.: PCT/AU2022/050235

§ 371 Date: Sep. 11, 2023

(30) Foreign Application Priority Data
Mar. 17, 2021 (AU) 2021900775

(51) Int. Cl. C12N 11/04; A01N 25/04; A01N 63/00; A01P 9/00; C12N 1/10; C12N 11/10

(52) U.S. Cl. CPC C12N 11/04; A01N 25/04; A01N 63/00; A01P 9/00; C12N 1/10; C12N 11/10

(57) ABSTRACT

The present disclosure relates to compositions and methods for inducing encystment of and/or stabilising ciliate cells. In particular, the present disclosure relates to polymers and polymeric solutions, including hydrocolloids, which may be used to induce encystment of trophont ciliate cells to form encysted ciliate cells and/or stabilise encysted cells. The present disclosure also relates to methods of infecting and/or colonising molluscs with the compositions and ciliates described herein.

### CROSS-REFERENCE TO RELATED APPLICATION

This application claims the benefit of Australia Patent Application No. 202190775 filed on Mar. 17, 2021, and PCT/AU2022/050235 filed Mar. 17, 2022, which are hereby incorporated by reference.

### FIELD OF THE INVENTION

The present disclosure relates to compositions and methods for inducing encystment of and/or stabilising ciliate cells. In particular, the present disclosure relates to polymers and polymeric solutions which may be used to induce encystment of trophont ciliate cells to form encysted ciliate cells and/or stabilise encysted cells. The present disclosure also relates to methods of infecting and/or colonising molluscs with the compositions and ciliates described herein.

### BACKGROUND OF THE INVENTION

Pests, for example slugs and snails, are a problem in agriculture and horticulture because they damage plants and affect the productivity and quality of crops and plant products. Various strategies have been used to control pest molluscs which include the use of chemical molluscicides (e.g. methiocarb and metaldehyde) which are usually distributed in baits. These chemicals are not just specific for molluscs, and target other animals raising concerns about their toxic effect and environmental contamination.

Biological agents, such as biological pesticides, have been proposed as an alternative to chemical molluscicides. One example is the slug parasitic ciliate Tetrahymena rostrata. T. rostrata ciliates transition from being feeding ciliated cells (called “trophont” cells) to reproductive or resting cyst cells (called “cyst” or “encysted” cells) via a process called “encystment”. The encysted cells then undergo “excystment” to form juvenile cells (called “theront” cells).

Various problems arise associated with using ciliate cells as biological pesticides due to their developmental life cycle, including that cells grown in culture media are not robust and/or have limited cell viability so cannot be stored for long periods of time thus are unsuitable for use as pest control agents. There is therefore a need to develop new processes to enhance the storage, stability, methods for application, and/or parasitic activity of ciliate cells for use in pest control, or at least provide the public with a useful alternative.

### SUMMARY OF THE INVENTION

The present inventors have identified processes for growing and formulating ciliate cells at various developmental stages for production, storage and delivery as a biological pesticide for the control of pests, such as molluscs. In particular, the present inventors have identified that various polymers improve the storage, stability and viability of ciliate cells. The polymers described herein can induce the encystment of trophont ciliate cells to form one or more encysted ciliate cells. The polymers can be provided as an aqueous polymer solution, which may be used to encyst or stabilise ciliate cells suspended therein or can be a dried polymer (e.g. via dehydration) wherein one or more encysted ciliate cells are interspersed therein. The polymers may be non-ionically crosslinked.

The encysted ciliate cells remain viable during storage either suspended in the polymeric solution or interspersed within the polymer following dehydration. Alternatively, the polymers described herein can be used to stabilise and store preformed encysted cells. The encysted ciliate cells can be subsequently released from the polymer and undergo excystment into theront ciliate cells, which the inventors have also identified can be highly infective to pests such as molluscs. The present invention therefore provides compositions which can be used to store, stabilise and transport viable encysted ciliate cells, allowing for their use as an effective pest control agent, such as being applied to areas affected or likely to be affected by a pest species.

In a first aspect, there is provided a composition comprising encysted ciliate cells that are interspersed within a polymer. The polymer may be a non-ionically crosslinked polymer.

In one embodiment, the polymer comprises a polysaccharide. In one embodiment, the polysaccharide is an anionic polysaccharide. In one embodiment, the polysaccharide is derived from one or more of algae, plants, animals, fungi, or bacteria. In one embodiment, the polysaccharide is derived from algae or bacteria. In one embodiment, the polysaccharide is an algal polysaccharide or a bacterial polysaccharide. In one embodiment, the polysaccharide is an algal polysaccharide. In one embodiment, the polysaccharide is a bacterial polysaccharide. In one embodiment, the polysaccharide is selected from the group consisting of carrageenan, agarose, agar, alginate, xanthan gum and gellan gum or a mixture thereof. In one embodiment, the polysaccharide is carrageenan, xanthan gum or gellan gum, or a mixture thereof. In one embodiment, the polysaccharide is carrageenan. In another embodiment, the polysaccharide is xanthan gum. In another embodiment, the polysaccharide is gellan gum.

In one embodiment, the polymer is dispersed in an aqueous solution, and the encysted ciliate cells are suspended in the aqueous solution comprising the polymer. Alternatively, in one embodiment, the polymer is a dried polymer. In one embodiment, the aqueous solution comprising the polymer forms a hydrocolloid. In some embodiments, the aqueous solution comprising the polymer forms an anionic hydrocolloid. The ciliate cells may be suspended within the hydrocolloid. In one embodiment, the aqueous solution comprises between about 0.01% w/v to about 5% w/v polymer based on the total volume of the aqueous solution. In one embodiment, the aqueous solution comprises between about 0.01% w/v to about 1% w/v polymer based on the total volume of the aqueous solution.

In one embodiment, the aqueous solution is a buffer solution. In one embodiment, the buffer solution comprises a HEPES buffering agent. In one embodiment, the concentration of the HEPES buffering agent in the buffer solution is between about 1 mM to about 25 mM. In one embodiment, the buffer solution has a pH of between about 6.0 to about 9.0.

In one embodiment, the encysted ciliate cells interspersed within the polymer remain viable for at least about four weeks.

In one embodiment, the ciliate cells are a member of the Ciliophora phylum. In one embodiment, the ciliate cells are a member of the Heterotrichea, Karyorelictea, Armophorea, Litostomatea, Colpodea, Nassophorea, Phyllopharyngea, Prostomatea, Plagiopylea, Oligohymenophorea, Protocruziea, Spirotrichea, or Cariotrichea class. In one embodiment, the ciliate cells are a member of the Apostomatia, Astomatia, Hymenostomatia, Peniculia, Peritrichia, or Scuticociliatia order. In one embodiment, the ciliate cells are a member of the Tetrahymenidae, Ophryoglenina, or Peniculina family. In one embodiment, the ciliate cells are a member of the Tetrahymena genus. In one embodiment, the ciliate cells are of the T. rostrata, T. hegewischi, T. hyperangularis, T. malaccensis, T. patula, T. pigmentosa, T. foissneri, T. deweyae T. pyriformis, T. thermophila, T. vorax, T. geleii, T. corlissi, T. empidokyrea or T. limacis species. In one embodiment, the ciliate cells are of the T. rostrata species.

In a related and second aspect, there is provided a method of inducing the encystment of ciliate cells, the method comprising incubating a population of trophont ciliate cells in an aqueous solution comprising a polymer at a temperature and for a period of time effective to induce the encystment of the trophont ciliate cells to form one or more encysted ciliate cells.

In a related and third aspect, there is provided a method of stabilising encysted ciliate cells, the method comprising suspending a population of encysted ciliate cells in an aqueous solution comprising a polymer. The embodiments described above for the polymer, aqueous solution and ciliate cells in relation to the composition comprising ciliate cells equally apply to the methods of encystment and stabilising of ciliate cells described herein.

In some embodiments, the aqueous solution comprising the polymer forms a hydrocolloid. In some embodiments, the aqueous solution comprising the polymer forms an anionic hydrocolloid. In one embodiment, the polymer is not ionically crosslinked during encystment.

In one embodiment, the trophont ciliate cells are incubated in the aqueous solution at a temperature of between about 10° C. to about 30° C. In one embodiment, the trophont ciliate cells are incubated in the aqueous solution for period of time of between about 12 to about 72 hours. In one embodiment, the encysted ciliate cells are stored in the aqueous solution at a temperature of between about 10° C. to about 30° C.

In some embodiments, the method further comprises the step of adding magnesium ions to the aqueous solution prior to or during incubation in an amount effective to stimulate encystment of one or more trophont ciliate cells. Alternatively or additionally, the method may further comprise the step of adding magnesium ions to the aqueous solution following incubation in an amount effective to stabilise the one or more encysted ciliate cells. In either embodiment, the magnesium ions may be provided by a suitable magnesium salt, for example magnesium sulfate.

In some embodiments, the method further comprises dehydrating the aqueous solution to obtain a dried polymer wherein the one or more encysted ciliate cells are interspersed within the polymer.

In a related and fourth aspect, there is provided an encystment media composition comprising an aqueous solution and a non-ionically crosslinked polymer. The embodiments described above for the polymer and aqueous solution in relation to the compositions comprising ciliate cells and methods of encystment and stabilising of ciliate cells methods equally apply to the encystment media composition described herein.

In a related and fifth aspect, there is provided an agricultural or horticultural composition comprising the composition as described above or an encysted ciliate cell prepared or stabilised by the method as described above, in combination with one or more agriculturally or horticulturally acceptable carriers.

In a related and sixth aspect, there is provided a method of infecting or colonising a pest species with a ciliate, the method comprising applying to an area affected or likely to be affected by a pest species a composition as described above or an encysted ciliate cell prepared or stabilised by the method as described above.

In a related and seventh aspect, there is provided a method of infecting or colonising a pest species with a ciliate, the method comprising applying a composition as described above or an encysted ciliate cell prepared or stabilised by the method as described above to the pest species.

In a related and eighth aspect, there is provided a method for reducing agricultural crop damage comprising applying to an agricultural crop area affected or likely to be affected by a pest species a pest species a composition as described above or an encysted ciliate cell prepared or stabilised by the method as described above.

In one embodiment, the method results in the ciliate killing or affecting the fitness of the pest species. In one embodiment, the pest species is an invertebrate. In one embodiment, the pest species is a mollusc. In one embodiment, the mollusc is a Gastropod. In one embodiment, the Gastropod is a snail or slug.

Any embodiment herein shall be taken to apply mutatis mutandis to any other embodiment unless specifically stated otherwise. For instance, the skilled person would understand that examples and embodiments of polymers, aqueous solutions, additives and ciliate cells described herein in relation to the composition comprising encysted ciliate cells equally apply to the method of inducing encystment and method of stabilising ciliate cells, encystment media compositions, agricultural or horticultural compositions, methods of infecting a pest species and/or methods for reducing agricultural crop damage.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

### BRIEF DESCRIPTION OF THE DRAWINGS

FIG. 1—Ciliate cell developmental stages: Schematic diagram of the main developmental stages of the ciliate T. rostrata depicting 1) trophont ciliate cells, 2) encysted ciliate cells and 3) theront ciliate cells. Trophont ciliate cells undergo “encystment” to form encysted ciliate cells (or “cysts”). Encysted ciliate cells undergo “excystment” to form theront ciliate cells. Theront ciliate cells undergo “maturation” to form trophont ciliate cells.

FIG. 2—Carrageenan induces encystment of T. rostrata at 26° C.: Percentage of encysted cells (% round) following incubation of trophonts in 0.25% w/v carrageenan buffer solution (0.25% w/v carrageenan-H; triangle and grey), 0.125% carrageenan buffer solution (0.125% carrageenan-H; square and light grey) and compost infusion (CI—H; circle and black) at 26° C. The corresponding dashed lines represent the viability of the encysted ciliate cells after 31 days incubation.

FIG. 3—Carrageenan induces encystment of T. rostrata at 20° C.: Percentage of encysted cells (% round) following incubation of trophonts in 0.25% w/v carrageenan buffer solution (0.25% w/v carrageenan-H; triangle and grey), 0.125% carrageenan buffer solution (0.125% carrageenan-H; square and light grey) and compost infusion (CI—H; circle and black) at 20° C. The corresponding dashed lines represent the viability of the encysted ciliate cells after 31 days incubation.

FIG. 4—Encysted ciliate cells suspended in carrageenan remain stable after 4 weeks storage: Encysted ciliate cells in 0.25% w/v carrageenan buffer solution (0.25% w/v carrageenan-H; triangle and grey), 0.125% carrageenan buffer solution (0.125% carrageenan-H; square and light grey) and compost infusion (CI—H; circle and black) were transferred and stored to 20° C. The corresponding dashed lines represent the viability of the encysted ciliate cells after 31 days storage at 20° C.

FIG. 5—Magnesium sulfate stabilises encysted ciliate cells in carrageenan: Survival of encysted ciliate cells in 0.25% w/v carrageenan buffer solution (0.25% w/v carrageenan-H; triangle and grey), 0.125% carrageenan buffer solution (0.125% carrageenan-H; square and light grey) and compost infusion (CI—H; circle and black) exposed to 50 mM magnesium sulfate after 24 hours incubation at 26° C. prior to transfer to 20° C. The corresponding dashed lines represent the viability of the encysted ciliate cells after 31 days storage at 20° C.

FIG. 6—Morphology of encysted ciliate cells in carrageenan: Micrograph of encysted ciliate cells formed in carrageenan having the distinctive capsules characteristic of typical T. rostrata cysts.

FIG. 7—Encysted ciliate cells in carrageenan tolerate dehydration: Micrograph of encysted ciliate cells in carrageenan following dehydration.

FIG. 8—Composted pine bark particles induce encystment of T. rostrata: Extracts of composted pine bark (CI, DCIP and DUP4) were added at various concentration to T. rostrata at 1-3×104 cells per ml (low cell density) in buffer to stimulate encystment at 26° C. After 26 hours the percent of cells that had encysted was determined.

FIG. 9—Carrageenan and gellan gum induce encystment of T. rostrata: Solutions of carrageenan buffered before and after (#) autoclaving and gellan gum (HA and LA) were added at various concentration to T. rostrata at 1-3×104 cells per ml (low cell density) in buffer to stimulate encystment at 26° C. After 26 hours the percent of cells that had encysted was determined.

FIG. 10—Morphology of encysted ciliate cells in compost infusion, MgSO4 and carrageenan: Giemsa stained theronts produced in buffered CI, MgSO4, 0.025% w/v carrageenan (CGN) and 0.125% w/v carrageenan (CGN) at 26° C./20° C. at a low cell density of 1-3×104 cells per ml.

FIG. 11—Carrageenan produces infections theronts: Trophonts cultures were encysted in encystment buffer with CI (circles), 62.5 uM MgSO4 (squares), 0.025% w/v carrageenan (CGN) (diamonds) and 0.125% w/v carrageenan (CGN) (triangles) at 26° C. resulting in cyst suspensions on days 1-3. The cysts were transferred to 20° C. and excystment of theronts was completed by day 7-8 and were then maintained under starvation conditions in the respective encystment buffer at 20° C. The average viable counts per ml are shown with maximum and minimal values of three separate culture. A summary timeline for encystment, excystment and theront persistence is given.

FIG. 12—Survival of slugs exposed to theronts produced in carrageenan: Survival of D. reticulatum slugs when challenge with theronts made in carrageenan, UP6 (extract of composted pine bark) and 62.5 uM MgSO4 buffer solutions

FIG. 13—Carrageenan and xanthan gum induce encystment of T. rostrata: Extracts of composted pine bark, carrageenan and xanthan gum were added at various concentration to T. rostrata at 1-3×104 cells per ml (low cell density) in buffer to stimulate encystment at 26° C. After 26 hours the percent of cells that had encysted was determined.

FIG. 14—High acyl gellan gum induce encystment of T. rostrata: Solutions of HA gellan gum, made with a heat (90-100° C. or 50-60° C.)—cooling (to 17-20° C.) step or without (17-20° C.), were added at various concentration to T. rostrata at 1-3×104 cells per ml (low cell density) in buffer to stimulate encystment at 26° C. After 26 hours, the percent of cells that had encysted was determined.

### DETAILED DESCRIPTION

### General Techniques and Selected Definitions

Unless specifically defined otherwise, all technical and scientific terms used herein shall be taken to have the same meaning as commonly understood by one of ordinary skill in the art (e.g., polymer, buffer, pest control, and ciliate physiology).

As used herein, the term “about”, unless stated to the contrary, refers to +/−10%, more preferably +/−5%, more preferably +/−1%, of the designated value.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The term “consists of”, or variations such as “consisting of”, refers to the inclusion of any stated element, integer or step, or group of elements, integers or steps, that are recited in context with this term, and excludes any other element, integer or step, or group of elements, integers or steps, that are not recited in context with this term.

As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Further, at least one of A and B and/or the like generally means A or B or both A and B. In addition, the articles “a” and “an” as used in this application and the appended claims may generally be construed to mean “one or more” unless specified otherwise or clear from context to be directed to a singular form.

The term “suspended”, “suspension” or “suspending” in the context of ciliate cells being suspended in an aqueous solution comprising a polymer, refers to a polymer solution that can flow but is sufficient to hold the ciliate cells in suspension. The ciliate cells can migrate throughout the polymer solution while remaining suspended.

The term “intersperse”, “interspersion” or “interspersed” in the context of ciliate cells being interspersed within a polymer, refers to ciliate cells being at least in part distributed and scattered within the polymer.

The term “incubate”, “incubating” or “incubated” in the context of ciliate cells being incubated in an aqueous solution, refers to maintaining ciliate cells under controlled conditions favourable for inducing encystment and/or maintaining viability of the ciliate cells.

The term “biological pesticide” refers to a pest management agent that is based on organisms or natural products that provide pest intervention through a predatory, parasitic or chemical relationship, often having a short term effect on the environment.

### Ciliate Cells

In some embodiments or examples, the methods and compositions of the present invention comprise the incubation/suspension/interspersion of ciliate cells within a polymer, for example an aqueous solution comprising the polymer or a dried polymer.

The term “ciliates” refers to a group of protozoans characterized by the presence of hair-like organelles called cilia. It is the presence of cilia which distinguishes ciliate cells from other protist cells. The developmental stage cycle of ciliate cells can be separated into three main stages, namely the formation of 1) “trophont” ciliate cells (also known as trophozoites), 2) “encysted” or “cyst” ciliate cells and 3) “theront” ciliate cells. Cells go through several stages of development during autogamy and cyst maturation. The ciliate cells may be ciliate protozoa cells.

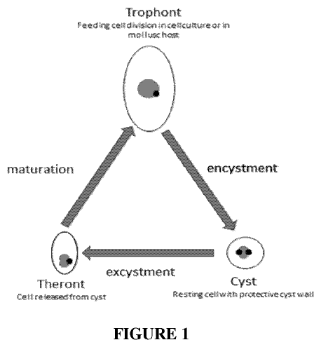

Briefly, the trophont cells are at the growing and feeding stage, encysted cells are the reproductive or resting stage, and theront cells are excysted cells. A simple summary of the life cycle of ciliate cells is provided in FIG. 1, where trophont cells undergo a process called “encystment” to form encysted cells. The encysted cells can then undergo a process called “excystment” to form theront cells. The developmental cycle closes where the theront cells mature (i.e. “maturation”) to form trophont cells. The encystment of trophont ciliate cells can be induced by various external stimuli such as starvation and cell aggregation. For example, trophonts respond to encystment stimuli by transforming into small, rapid swimming pre-cystic cells and then round up and secrete large amounts of mucin which condenses and gradually forms a laminar cyst wall which develops into the thick wall of encysted ciliate cells.

The present inventors have identified that, in some embodiments, polymers, such as polysaccharides, can induce encystment of trophont ciliate cells into encysted ciliate cells. The encysted ciliate cells incubated/suspended in the aqueous solution remain stable and viable and do not excyst into theront ciliate cells, even after dehydration to form a dried polymer wherein the encysted ciliate cells are interspersed within the polymer. In some embodiments, theront ciliate cells may be incubated/suspended/interspersed within the aqueous solution. Accordingly, it will be appreciated that a ciliate cell from any developmental stage may be used in the methods and compositions of the present invention.

In some embodiments, the ciliate cells may be encysted ciliate cells or trophont ciliate cells. In one preferred embodiment, the ciliate cells are trophont ciliate cells. In an alternative embodiment, the ciliate cells are encysted ciliate cells. In some embodiments, the ciliate cells are excysted ciliate cells. In one embodiment, the ciliate cells are theront cells. In some embodiments, the ciliate cells may be a mixture of trophont ciliate cells or encysted ciliate cells. In one embodiment, the trophont ciliate cell may undergo encystment within the aqueous solution to form an encysted ciliate cell. The present inventors have found that, in some embodiments, ciliate cells remained stable and viable for a longer period of time in the presence of polymers described herein.

The ciliate cells may be any member of the Ciliophora phylum. In one preferred embodiment, the ciliate cells are cells that are capable of encystment. The specific type of ciliate cell that is used may also depend on a number of variables, including but not limited to, the type of polymer and/or aqueous solution, the area to be treated with polymer, the soil type the polymer is being dispersed in, and/or the pest species being targeted for pest control (e.g. the type of pest, such as a Gastropod).

In some embodiments, the ciliate cell is a member of the Intramacronucleata, Ventrata, Spirotrichia, or Rhabdophora subphylum. In one preferred embodiment, the ciliate cell is a member of the Intramacronucleata subphylum.

In some embodiments, the ciliate cell is a member of Heterotrichea, Karyorelictea, Armophorea, Litostomatea, Colpodea, Nassophorea, Phyllopharyngea, Prostomatea, Plagiopylea, Oligohymenophorea, Protocruziea, Spirotrichea, or Cariotrichea class. In a preferred embodiment, the ciliate cells are a member of the Oligohymenophorea class.

In some embodiments, the ciliate cells are a member of the Apostomatia, Astomatia, Hymenostomatia, Peniculia, Peritrichia, or Scuticociliatia order. In a preferred embodiment, the ciliate cells are a member of the Hymenostomatia order.

In some embodiment, the ciliate cells are a member of the Tetrahymenina, or Ophryoglenia sub order. In one embodiment, the ciliate cells are a member of the Tetrahymenina sub order. In another embodiment, the ciliate cells are a member of the Ophryoglenia sub order.

In some embodiments, the ciliate cells are a member of the Tetrahymenidae, Ophryoglenina, or Peniculina family.

In some embodiments, the ciliate cells are a member of the Tetrahymena or Lambornella genus. In one embodiment, the ciliate cells are a member of the Lambornella genus. In a preferred embodiment, the ciliate cells are a member of the Tetrahymena genus.

In some embodiments, the ciliate cells are of the T. rostrata, T. hegewischi, T. hyperangularis, T. malaccensis, T. patula, T. pigmentosa, T. foissneri, T. deweyae, T. pyriformis, T. thermophila, T. vorax, T. geleii, T. corlissi, T. empidokyrea or T. limacis. In a preferred embodiment, the ciliate cells is of the T. rostrata species. In other embodiments, the ciliate cells are of the Lambornella clarki species.

In one embodiment, the ciliate cells are cultured from an isolated strain of T. rostrata TRAUS (deposited under PTA-126056 on 13 Aug. 2019 at the American Type Culture Collection).

It will be appreciated that the embodiments described above and herein in relation to the ciliate cells equally to the ciliate cells of the encystment methods, stabilisation methods, and encystment media composition as described herein.

### Compositions Comprising Encysted Ciliate Cells

The present inventors have identified a composition comprising encysted ciliate cells that are interspersed within a polymer can improve the storage, stability and viability of ciliate cells. In some embodiments, the polymer of the composition may be provided as an aqueous polymer solution or as a dried polymer. The polymer may be a hydrocolloid as described herein. The polymer and aqueous solution comprising a polymer are described herein.

### Polymers

The polymer is not lethal to ciliate cells, and allows sufficient diffusion of oxygen to the ciliate cells incubated/suspended therein to maintain cell viability.

In one embodiment, the polymer is a hydrophilic polymer. In some embodiments, the polymer may be selected hydrophilic acrylics, peptides, dendrimers, star-polymers, aliphatic polymers, natural polymers, polysaccharides, synthetic polymers, anionic polymers, cationic polymers, neutral polymers, and synthetic polymers, and mixtures or co-polymers thereof. In one embodiment, the polymer is an anionic polymer.

The polymer may have a high molecular weight (Mw). In some embodiments, the polymer has an average molecular weight (MW) in the range of between about 100 to about 1,000 kDa. In some embodiments, the polymer has an average molecular weight (MW) of at least about 100, 200, 300, 400, 500, 600, 700, 800 900 or 1,000 kDa. In other embodiments, the polymer has an average molecular weight (MW) of less than about 1,000, 900, 800, 700, 600, 500, 400, 300, 200 or 100 kDa. Combinations of these molecular weights to form various ranges are also possible, for example the polymer has an average molecular weight (MW) of between about 200 kDa to about 800 kDa. It will be appreciated that the molecular weight of the polymer may vary depending on the type used to prepare the aqueous solution (e.g. hydrocolloid). For example, different grades of carrageenan can be used.

In one embodiment, the polymer comprises a polysaccharide (i.e. a carbohydrate polymer). A polysaccharide is a polymeric carbohydrate comprising two or more monosaccharide units bound together by glycosidic linkages. Polysaccharides are widespread in nature and can be found in animals, plants, and bacteria, and can also be synthetically manufactured. In some embodiments, the polysaccharide may be a naturally occurring polysaccharide (e.g. derived from bacteria or algae such as xanthan gum, gellan gum or carrageenan) or a synthetic polysaccharide (e.g. CMC), or a mixture thereof.

According to at least some embodiments or examples described herein, the present inventors have surprisingly identified that polysaccharides can induce encystment of trophont ciliate cells to form encysted ciliate cells, which remain encysted and viable during storage. Without wishing to be bound by theory, it is believed that the presence of hydroxyl (—OH) groups along with the presence of various hydrophilic groups (e.g. —NH2, —COOH, —OH, —CONH2, —CONH—, and —SO3H) within polysaccharides, such as sulphate groups on carrageenan and carboxyl groups on the gellan gum and xanthan gum, can increase the affinity for binding to water molecules of the aqueous solution and capture of water to form a hydrocolloid, and in turn effectively dehydrates the trophont cells suspended within the polysaccharide solution, thus inducing encystment. Alternatively or additionally, the complex structure and hydrocolloid formation of various polysaccharides when dissolved in water may cause mechanical stress inducing encystment due to crowding the ciliate cells. Depending on the nature of the polysaccharide and/or process conditions used to prepare the aqueous hydrocolloid (e.g. heat treatment and cooling), the internal structure of the hydrocolloid may vary from randomly arranged polysaccharide subunits such as linear or coiled domains to a more ordered structure such as double helices or matrices. These double helices or matrices may further aggregate into a more complex hydrocolloid structure. By manipulating the internal structure of the hydrocolloid (e.g. via heat treatment and cooling of the polysaccharide aqueous solution prior to incubation), encystment efficiency may be improved. Alternatively or additionally, the hydrocolloid molecules may interact with one or more ligands on the surface of the ciliate cells to stimulate an encystment response, including for example interaction with one or more surface-exposed glycoproteins.

In one embodiment, the polysaccharide is derived from one or more of algae, plants, animals, fungi, or bacteria. In one embodiment, the polysaccharide is an algal polysaccharide or a bacterial polysaccharide.

In one embodiment, the plant polysaccharide (e.g. derived from plants) is selected from the group consisting of cellulose, starch, guar gum, pectin, locust bean gum, gum arabic, tragacanth and karaya, or a mixture thereof.

In one embodiment, the algal polysaccharide (e.g. derived from algae) is selected from the group consisting of carrageenan, agarose, agar and alginate, or a mixture thereof. In one embodiment, the polysaccharide is carrageenan.

In one embodiment, the animal derived polysaccharide is chitosan (e.g. from crustacean shells), chitin or gelatin, or a mixture thereof. In one embodiment, the fungal polysaccharide is galactomannan.

In one embodiment, the bacterial polysaccharide may be an extracellular polysaccharide. The extracellular polysaccharide may be selected from the group consisting of xanthan gum, gellan gum (including derivatives such as sphingans, welan, rhamsan and diutan), bacterial alginate, glucans (including alpha-glucans such as dextran, mutan, alternan and reuteran, and beta-glucans, such as cellulose and curdlan), hyaluronan, succinoglycan, levan, GalactoPol and FucoPol.

It will be appreciated that the polysaccharides may be obtained by any suitable method. Some non-limiting examples include polysaccharides obtained from a commercial supplier, or polysaccharides extracted from soil particles (including for example composted or non-composted particles, such as pine bark, peat or sugarcane bagasse and other composted or non-composted plant substrates). For example and in some embodiments, bacterial polysaccharides as described herein may be derived from composted pine bark particles. In some embodiments, bacterial polysaccharides derived from the composted pine bark particles may be harvested from the composted pine bark (e.g. isolated from the composted pine bark), and subcultured separately and added to the aqueous solution. Alternatively, the composted pine bark particles may be included as an additive which is suspended in the aqueous solution, wherein the bacterial polysaccharide (or other metabolite) is derived therefrom. The aqueous solution described herein (including in relation to the compositions, encystment media and methods of inducing encystment) may further comprise composted pine bark particles. The composted pine bark particles are described herein, including under the heading “Additives”.

The term “composted” refers to particles, such as pine park particles, that have been obtained through a composting process, that is have undergone microbial decomposition. By way of example only, composted pine bark particles may be obtained by adding urea (and optionally some trace elements) to saturated pine bark particles. It will be appreciated that the pine bark is the major carbon source and urea is the source of nitrogen, which together triggers the bacterial decomposition of the pine bark particles to obtain the “composted” pine bark particles. Alternatively, the “composted” pine bark particles may be obtained from a suitable potting mix. The term “potting mix” refers to a growing mix often used in gardens and nurseries, which comprise composted pine bark particles and other ingredients such as sand, added nutrients and other additives such as vermiculite. An example of a potting mix is Australian Growing Solutions. The potting mix may be filtered to obtain the composted pine bark particles. The pine bark particles may comprise Pinus radiata pine particles. In some embodiments, adding composted pine bark particles to the aqueous solution introduces the bacterial polysaccharides into the aqueous solution.

In other embodiments, the bacterial polysaccharides as described herein may be derived from non-composted particles. For example, non-composted particles such as pine bark particles or sugarcane bagasse particles, may be subjected to conditions effective to encourage microbial growth on the particles, e.g. the particles undergo a microbial fermentation or culturing under conditions effective to encourage microbial growth on the particle surface (e.g. maceration). Alternatively, non-composted particles may be fermented with a defined culture of one or more bacteria to provide a non-composted particle preparation that provides on or more bacterial polysaccharides that can induce encystment. In this embodiment, it would be appreciated that growing bacterial polysaccharides on a plant material requires a nitrogen source (e.g. urea) and optionally a buffer such as a phosphate buffer, followed by removal the larger particles to obtain the bacterial polysaccharide. For example, xanthan gum may be produced by culturing Xanthomonas campestris on non-composted particles such as sugarcane bagasse, followed by removal of the large particles to obtain xanthan gum, which can be precipitated to obtain small particles comprising the xanthan gum, which can be dried and then rehydrated for later use in encystment. In one embodiment, the bacterial polysaccharide is isolated from composted pine bark or fermented sugarcane bagasse.

In one embodiment, the polysaccharide may be a synthetic polysaccharide, including synthetic versions of one or more of the polysaccharides described herein. Examples of suitable synthetic polysaccharides include carboxymethyl cellulose (CMC). In one embodiment, the polysaccharide is an anionic polysaccharide. The term “anionic” in the context of a polysaccharide refers to a polysaccharide comprising one or more anionic groups (e.g. sulfate; SO32- or carboxylate; COO—). The anionic nature of the polysaccharide may be controlled by the environment, for example by controlling the pH of the aqueous solution described herein (e.g. suspending the polysaccharide in an aqueous buffer solution (e.g. HEPES buffer pH 7). According to some embodiments or examples described herein, the present inventors have surprisingly identified that anionic polysaccharides can induce encystment of trophont ciliate cells to form encysted ciliate cells which remain encysted and viable during storage. Without wishing to be bound by theory, it is believed that the polysaccharides anionic groups may interact with one or more ligands on the surface of T. rostrata trophont ciliate cells, including for example interaction with one or more surface-exposed glycoproteins, which may help stimulate encystment, for example either by aggregating the cells and/or stimulating capsule formation. For example, the sulfate (SO32-) groups on carrageenan, and carboxylate (COO—) groups on gellan gum have a strong negative charge and affinity to polycation macromolecule and/or amphoteric proteins on the surface of ciliate cells. The resulting interaction may be sensed by the cell and trigger encystment.

In some embodiments, the polysaccharide may be selected from the group consisting of carrageenan, agarose, agar, alginate, alginic acid, carboxymethylcellulose, pectin, pectic acid, hyaluronic acid, polyglucuronic acid, cellulose, gellan gum, xanthan gum, starch, chitosan, and chitin, or mixtures or co-polymers thereof. In one embodiment, the polymer comprises a polysaccharide selected from the group consisting of carrageenan, agarose, gellan gum, xanthan gum and agar, or a mixture thereof. In one embodiment, the polymer comprises a polysaccharide selected from the group consisting of carrageenan, gellan gum and xanthan gum, or a mixture thereof.

It will be appreciated that the polymer may comprise any one or more of the above mentioned polymers (e.g. polysaccharides), including synthetic versions, mixtures or copolymers thereof.

In one embodiment, the polymer is carrageenan. Carrageenan is a sulfated polysaccharide, namely a sulfated galactan that is extracted from seaweed. Carrageenan comprises an anionic linear polymers comprising 1,3α-1,4β-galactans having one (κ-) two (τ-) or three (λ-) sulfates per disaccharide unit.

The present inventors have identified that, in some embodiments, carrageenan can induce encystment of trophont ciliate cells to form encysted ciliate cells which remain encysted and viable during storage. Alternatively, carrageenan can be used to suspend and store preformed encysted ciliate cells. The encysted ciliate cells can excyst to form theront cells once released from the carrageenan and can infect pests such as molluscs.

In some embodiments, the carrageenan comprises a mixture of κ-carrageenan, τ-carrageenan, and λ-carrageenan. The carrageenan may comprise predominantly of κ-carrageenan (e.g. one sulfate per disaccharide unit). In one embodiment, the polymer is carrageenan purchased from Sigma Aldrich, catalogue number C1013 (CAS Number 9000-07-1). The carrageenan may comprise a small amount of potassium cations, for example less than about 15%, 10%, 5%, 4%, 3%, 2%, 1%, 0.5%, 0.1%, 0.01%, 0.001%, or 0.0001% w/w potassium cations.

The aqueous solution comprising carrageenan can form a hydrocolloid. Without wishing to be bound by theory, it is believed that solubilised carrageenan chains change conformation to from being random coils to a more ordered helical structure, which can then aggregate via weak intermolecular interactions, namely hydrogen bonding, between the carrageenan helices to form the hydrocolloid. The carrageenan may also comprise a small fraction of potassium (K+) cations, the presence of which can stabilize the junction zones between two carrageenan helices by binding to the negatively charged sulfate groups without hindering the hydrogen bonding of the two helices. Importantly, the stabilisation of junction zones between carrageenan helices by monovalent potassium cations does not result in ionic crosslinking thus retaining the hydrocolloids water-soluble properties.

It will be appreciated that carrageenan has a sulfate content (SO3-). In some embodiments, the polymer has a sulfate content of between about 1% to about 50% w/w sulfate based on the total mass of the polymer. In some embodiments, the polymer has a sulfate content of at least about 1, 2, 5, 10, 15, 20, 25, 30, 40 or 50% w/w sulfate based on the total weight of the polymer. In some embodiments, the polymer has a sulfate content of less than about 50, 40, 30, 25, 20, 15, 10, 5, 2 or 1% w/w sulfate based on the total weight of the polymer. Combinations of any two of these upper or lower values to form a range is also possible, for example between about 5% to about 40% w/w, or between about 10% to about 30% w/w sulfate based on the total weight of the polymer.

In another embodiment, the polysaccharide is gellan gum. Gellan gum is a bacterial polysaccharide and, like carrageenan, is a linear polymer which can act as a hydrocolloid. Gellan gum comprises tetrasaccharide repeats, 1,4-α-L-rhamnose, 1,3-β-D-glucose, 1,4-β-D-glucose, 1,4-β-D-glucuronic acid. Gellan gum comprises two acyl substituents, acetate and glycerate, which occur on the same glucose subunit. Depending on the number of acyl substituents, gellan gum can exist in either high acyl (HA), native, or low acyl (LA) forms. In one embodiment, the polysaccharide is a high acyl (HA) gellan gum (Kelcogel® LT100 HA gellan gum). In a related embodiment, the gellan gum has an acyl content (e.g. the number of acyl groups attached to the polysaccharide) of at least 25%, 30%, 35%, 40%, 45% or 50% w/w based on the total weight of the gellan gum. According to some embodiments or examples described herein, a gellan gum hydrocolloid stimulates encystment of T. rostrata, with HA gellan gum capable of producing durable cysts. In some embodiments, gellan gum that has not been heat-treated may induce encystment with high efficiency. Without wishing to be bound by theory, in relation to gellan gum it is believed the gellan gum polymers within the hydrocolloid interact in an irregular matrix structure resulting in a higher viscosity compared to gellan gum which has been heat treated and cooled which results in a softer, lower viscosity hydrocolloid. Nonetheless, both heat-treated and non-heat treated gellan gum was found to induce encystment.

In another embodiment, the polysaccharide is xanthan gum. Xanthan gum is a hetero-polysaccharide produced by the fermentation of the bacterial microbe Xanthomonas campestris. Its main chain is made up of glucose units. The beta-D-glucoses are linked (1->4) to form the backbone similar to cellulose. At every second glucose residue is an added trisaccharide side chain consisting of alpha-D-mannose, beta-D-glucuronic acid, and a beta-D-mannose terminal unit. Thus, the overall repeating structure is a pentasaccharide. The mannose residue closer to the backbone often has an acetyl group at its C-6 position, the more distant mannose residue often has a pyruvate group between its C-4 and C-6 atom. In general, about one branch in two has a pyruvate group, but the ratio of pyruvate to acetate varies depending on the substrain of Xanthomonas campestris used and the conditions of fermentation. The overall repeating structure is a pentasaccharide. The glucuronic and pyruvic acid groups give xanthan gum a highly negative charge.

In one preferred embodiment, the polymer is not ionically crosslinked. As used herein, the term “crosslink, “crosslinked” or “crosslinking” refers to the formation of interactions within or between polymers which result in the formation of a three-dimensional matrix. An ionically cross linked polymer is linked together by ionic interactions (i.e. an electrostatic attraction between oppositely charged ions). For example, ionic-cross linking may be a charge interaction between the polymer and an oppositely charged molecule as the linker, such as may be a cation or anion, as described in Parhi et al. (2017). In relation to this embodiment, it will be understood that there are no ionic cross-linking interactions which result in the formation of a three-dimensional polymer matrix, for example a hydrogel. The term “hydrogel” refers to a substance formed when a hydrogel-forming polymer (e.g. natural or synthetic polymer) is cross-linked (e.g. via ionic crosslinking) to create a three-dimensional “solid” matrix structure which entraps water molecules to form a gel. Consequently, where the polymer is part of an aqueous solution (e.g. suspended or dissolved in an aqueous buffer solution), it will be understood that the absence of any ionic crosslinking means the polymer remains suspended and dispersed within the aqueous solution and does not form a three-dimensional polymer matrix, such as a hydrogel. In one embodiment, the composition is not a hydrogel. In one embodiment, the composition is not an alginate hydrogel.

The lack of ionic crosslinking does not preclude the presence of weaker interactions between the polymer and water molecules, for example other types of physical cross-linking such as hydrogen bonding which, according to some embodiments or examples described, can facilitate the formation of a hydrocolloid. Examples of non-ionic physical cross-linking includes molecular entanglement of the polymer, hydrogen bonding and hydrophobic interaction.

In a related embodiment, the composition does not comprise an effective amount of an ionic cross-linker, for example a multivalent metal cation or multivalent anion, in an amount effective to ionically crosslink the polymer to form a hydrogel (e.g. capable of crosslinking the polymer to form a hydrogel as defined above).

It will be appreciated that the embodiments described above and herein in relation to the polymer apply equally to the polymers of the encystment methods, stabilisation methods, and encystment media composition as described herein.

### Aqueous Solution

The polymer may be dispersed in an aqueous solution. The encysted ciliate cells may be interspersed/suspended in the aqueous solution comprising the polymer.

The polymer may be dispersed in an aqueous solution. The aqueous solution comprising the polymer may also be called an aqueous polymer solution. In one embodiment, the aqueous solution comprising the polymer forms a hydrocolloid. The ciliate cells may be suspended within the hydrocolloid. The hydrocolloid may be an anionic hydrocolloid (e.g. derived from an anionic polymer). A “hydrocolloid” can be defined as a water-soluble hydrophilic polymeric that interacts with an aqueous medium (e.g. water) to form a colloid system either as a gel or a sol of solubilised hydrophilic polymer particles. The colloid particles are hydrophilic polymers (e.g. polysaccharides) dispersed in water resulting in a dispersion, and in some cases a viscous dispersion and/or gel. In the case of polysaccharides, the presence of hydroxyl (—OH) groups can increase the affinity for binding to water molecules of the aqueous solution and/or to each other, for example via weak non-ionic interactions (e.g. hydrogen bonding), resulting in a dispersion (i.e. an intermediate between a true solution and a suspension) and thus exhibits the properties of a colloid. In contrast, a hydrogel is three-dimensional “solid” matrix structure formed by a cross-linked hydrophilic polymer, the result of which is insoluble in water. The insoluble cross-linked hydrophilic polymer entraps water molecules (as opposed to being solubilised therein) within the cross-linked matrix structure to form a continuous gel network and is distinct from a hydrocolloid. In one embodiment, the composition is not a hydrogel. In one embodiment, the aqueous solution does not comprise a hydrogel. In one embodiment, the aqueous solution does not comprise an alginate hydrogel.

The aqueous solution may be water or may be a buffered solution. As used herein, the term “buffer solution” refers to an aqueous solution consisting of a mixture of a weak acid and its conjugate base, or vice versa. The pH of the buffer solution changes very little when a small amount of strong acid or base is added to it. Buffer solutions are used as a means of keeping pH at a nearly constant value. The buffer solution may also be referred to as an aqueous buffer solution.

The buffer solution may be a HEPES (4-(2-hydroxyethyl)-1-piperazineethanesulfonic acid), a phosphate buffer solution, or a Tris (tris(hydroxymethyl)aminomethane) buffer solution. In one embodiment, the buffer solution is a HEPES (4-(2-hydroxyethyl)-1-piperazineethanesulfonic acid) buffer solution.

In some embodiment, the concentration of HEPES in the HEPES buffer solution is at least about 1, 2, 5, 7, 10, 12, 15, 20 or 25 mM. In some embodiments, the concentration of HEPES in the HEPES buffer solution is less than about 25, 20, 15, 12, 10, 7, 5, 2 or 1 mM. Combinations of these concentrations are also possible, for example between about 1 mM to about 25 mM, between about 5 mM to about 25 mM, between about 8 mM to 15 mM, for example about 10 mM.

In some embodiments, the buffer solution has a pH of at least about 6, 6.2, 6.4, 6.6, 6.8, 7, 7.2, 7.4, 7.6, 7.8, 8, 8.2, 8.4, 8.6, 8.8 or 9. In some embodiments, the buffer solution has a pH of less than about 9, 8.8, 8.6, 8.4, 8.2, 8, 7.8, 7.6, 7.4, 7.2, 7, 6.8, 6.4, 6.2, or 6. Combinations of these pH values are also possible, for example between about 6.8 to about 8.2. In one embodiment, the buffer solution has a pH of about 7. The pH of the buffer solution may be adjusted using sodium hydroxide.

In some embodiments, the concentration of the polymer in the aqueous solution is between about 0.01% w/v to about 20% w/v based on the total volume of the aqueous solution. In some embodiments, the concentration of the polymer in the aqueous solution is at least about 0.01%, 0.015%, 0.02%, 0.025%, 0.05%, 0.1%, 0.2%, 0.3%, 0.4% 0.5%, 0.6% 0.7%, 0.8%. 0.9%, 1%, 1.1%, 1.2%, 1.3%, 1.4%, 1.5%, 2%, 2.5%, 3%, 3.5%, 4%, 4.5%, 5%, 10%, 15%, or 20% w/v based on the total volume of the aqueous solution. In other embodiments, the concentration of the polymer in the aqueous solution is less than about 20%, 15%, 10%, 5%, 4.5%, 4%. 3.5%, 3%, 2.5%, 2%, 1.5%, 1.4%, 1.3%, 1.2%, 1.1%, 1%, 0.9%, 0.8%, 0.7%, 0.6%, 0.5%, 0.4%, 0.3%, 0.2%, 0.1%, 0.05%, 0.025%, 0.02%, 0.015%, or 0.01% w/v based on the total volume of the aqueous solution. Combinations of any two of these upper or lower values to form a range are also possible, for example the concentration of the polymer in the aqueous solution is between about 0.01% w/v to about 15% w/v, between about 0.01% w/v to about 10% w/v, between about 0.01% w/v to about 5% w/v, or between about 0.01% w/v to about 1% w/v based on the total volume of the aqueous solution.

The concentration of the polymer in the aqueous solution may also be provided in mg/mL based on the total volume of the aqueous solution. In some embodiments, the concentration of polymer in the aqueous solution (in mg/mL) is at least about 0.001, 0.002, 0.005, 0.01, 0.02, 0.05, 0.1, 0.125, 0.15, 0.175, 0.2, 0.25, 0.5, 0.75, 1, 1.25, 1.5, 1.75, 2, 2.25, 2.5, 2.75, 3, 3.5, 4, 4.5 or 5. In some embodiments, the concentration of polymer in the aqueous solution (in mg/ml) may be less than about 5, 4.5, 4, 3.5, 3, 2.75, 2.5, 2.25, 2, 1.75, 1.5, 1.25, 1, 0.75, 0.5, 0.25, 0.2, 0.175, 0.15, 0.125, 0.1, 0.05, 0.02, 0.01, 0.005, 0.002, 0.001. Combinations of any two of these upper or lower values to form a range are also possible, for example the concentration of the polymer in the aqueous solution is between about 0.002 mg/mL to about 2.5 mg/mL.

In some embodiments, the concentration of the polymer in the aqueous solution is least about 0.01%, 0.015%, 0.02%, 0.025%, 0.05%, 0.1%, 0.15% 0.2%, 0.25%, 0.3%, 0.35%, 0.4%, 0.45%, 0.5%, 1% or 5% w/v based on the total volume of the aqueous solution. In other embodiments, the concentration of the polymer in the aqueous solution is less than about 5%, 1%, 0.5%, 0.45%, 0.4%, 0.35%, 0.3%, 0.25%, 0.2%, 0.15%, 0.1%, 0.05%, 0.025%, 0.02%, 0.015%, or 0.01% w/v based on the total volume of the aqueous solution. Combinations of any two of these upper or lower values to form a range are also possible, for example the concentration of the polymer in the aqueous solution is between about 0.01% w/v to about 0.5% w/v, between about 0.01% w/v to about 0.3% w/v based on the total volume of the aqueous solution. In some embodiments, the concentration of the polymer in the aqueous solution is about 0.01%, 0.015%, 0.02%, 0.025%, 0.05%, 0.1%, 0.15% 0.2%, 0.25%, 0.3%, 0.35%, 0.4%, 0.45%, or 0.5% w/v based on the total volume of the aqueous solution. It will be appreciated that the above % w/v concentrations apply to any polymer described herein, including those listed above under the heading “Polymer”.

In one embodiment, the polymer is a polysaccharide. In some embodiments, the concentration of polysaccharide in the aqueous solution is between about 0.01% w/v to about 20% w/v based on the total volume of the aqueous solution. In some embodiments, the concentration of polysaccharide in the aqueous solution is at least about 0.01%, 0.015%, 0.02%, 0.025%, 0.05%, 0.1%, 0.15%, 0.2%, 0.25%, 0.3%, 0.4% 0.5%, 0.6% 0.7%, 0.8%. 0.9%, 1%, 1.1%, 1.2%, 1.3%, 1.4%, 1.5%, 2%, 2.5%, 3%, 3.5%, 4%, 4.5%, 5%, 10%, 15%, or 20% w/v based on the total volume of the aqueous solution. In other embodiments, the concentration of polysaccharide in the aqueous solution is less than about 20%, 15%, 10%, 5%, 4.5%, 4%. 3.5%, 3%, 2.5%, 2%, 1.5%, 1.4%, 1.3%, 1.2%, 1.1%, 1%, 0.9%, 0.8%, 0.7%, 0.6%, 0.5%, 0.4%, 0.3%, 0.25%, 0.2%, 0.15%, 0.1%, 0.05%, 0.025%, 0.02%, 0.015%, or 0.01% w/v based on the total volume of the aqueous solution. Combinations of any two of these upper or lower values to form a range are also possible, for example the concentration of polysaccharide in the aqueous solution is between about 0.01% w/v to about 15% w/v, between about 0.01% w/v to about 10% w/v, 0.01% w/v to about 5% w/v, or between about 0.01% w/v to about 1% w/v based on the total volume of the aqueous solution.

In some embodiments, the concentration of polysaccharide in the aqueous solution is least about 0.01%, 0.015%, 0.02%, 0.025%, 0.05%, 0.1%, 0.15% 0.2%, 0.25%, 0.3%, 0.35%, 0.4%, 0.45%, 0.5%, 1% or 5% w/v based on the total volume of the aqueous solution. In other embodiments, the concentration of polysaccharide in the aqueous solution is less than about 5%, 1%, 0.5%, 0.5%, 0.45%, 0.4%, 0.35%, 0.3%, 0.25%, 0.2%, 0.15%, 0.1%, 0.05%, 0.025%, 0.02%, 0.015%, or 0.01% w/v based on the total volume of the aqueous solution. Combinations of any two of these upper or lower values to form a range are also possible, for example the concentration of polysaccharide in the aqueous solution is between about 0.01% w/v to about 0.5% w/v, between about 0.01% w/v to about 0.3% w/v based on the total volume of the aqueous solution. In some embodiments, the concentration of polysaccharide in the aqueous solution is about 0.01%, 0.015%, 0.02%, 0.025%, 0.05%, 0.1%, 0.15% 0.2%, 0.25%, 0.3%, 0.35%, 0.4%, 0.45%, or 0.5% w/v based on the total volume of the aqueous solution.

The concentration of polysaccharide in the aqueous solution may also be provided in mg/mL based on the total volume of the aqueous solution. In some embodiments, the concentration of polysaccharide in the aqueous solution (in mg/mL) is at least about 0.001, 0.002, 0.005, 0.01, 0.02, 0.05, 0.1, 0.125, 0.15, 0.175, 0.2, 0.25, 0.5, 0.75, 1, 1.25, 1.5, 1.75, 2, 2.25, 2.5, 2.75, 3, 3.5, 4, 4.5 or 5. In some embodiments, the concentration of polysaccharide in the aqueous solution (in mg/ml) may be less than about 5, 4.5, 4, 3.5, 3, 2.75, 2.5, 2.25, 2, 1.75, 1.5, 1.25, 1, 0.75, 0.5, 0.25, 0.2, 0.175, 0.15, 0.125, 0.1, 0.05, 0.02, 0.01, 0.005, 0.002, 0.001. Combinations of any two of these upper or lower values to form a range are also possible, for example the concentration of the polysaccharide in the aqueous solution is between about 0.002 mg/mL to about 2.5 mg/mL.

In one embodiment, the polymer is carrageenan. In some embodiments, the concentration of carrageenan in the aqueous solution is between about 0.01% w/v to about 20% w/v based on the total volume of the aqueous solution. In some embodiments, the concentration of carrageenan in the aqueous solution is at least about 0.01%, 0.015%, 0.02%, 0.025%, 0.5%, 0.1%, 0.15%, 0.2%, 0.3%, 0.4% 0.5%, 0.6% 0.7%, 0.8%. 0.9%, 1%, 1.1%, 1.2%, 1.3%, 1.4%, 1.5%, 2%, 2.5%, 3%, 3.5%, 4%, 4.5%, 5%, 10%, 15%, or 20% w/v based on the total volume of the aqueous solution. In other embodiments, the concentration of carrageenan in the aqueous solution is less than about 20%, 15%, 10%, 5%, 4.5%, 4%. 3.5%, 3%, 2.5%, 2%, 1.5%, 1.4%, 1.3%, 1.2%, 1.1%, 1%, 0.9%, 0.8%, 0.7%, 0.6%, 0.5%, 0.4%, 0.3%, 0.2%, 0.15%, 0.1%, 0.05%, 0.025%, 0.02%, 0.015%, or 0.01% w/v based on the total volume of the aqueous solution. Combinations of any two of these upper or lower values to form a range are also possible, for example the concentration of carrageenan in the aqueous solution is between about 0.01% w/v to about 15% w/v, between about 0.01% w/v to about 10% w/v, 0.01% w/v to about 5% w/v, or between about 0.01% w/v to about 1% w/v based on the total volume of the aqueous solution.

In some embodiments, the concentration of carrageenan in the aqueous solution is least about 0.01%, 0.015%, 0.02%, 0.025%, 0.05%, 0.1%, 0.15% 0.2%, 0.25%, 0.3%, 0.35%, 0.4%, 0.45%, 0.5%, 1% or 5% w/v based on the total volume of the aqueous solution. In other embodiments, the concentration of carrageenan in the aqueous solution is less than about 5%, 1%, 0.5%, 0.5%, 0.45%, 0.4%, 0.35%, 0.3%, 0.25%, 0.2%, 0.15%, 0.1%, 0.05%, 0.025%, 0.02%, 0.015%, or 0.01% w/v based on the total volume of the aqueous solution. Combinations of any two of these upper or lower values to form a range are also possible, for example the concentration of carrageenan in the aqueous solution is between about 0.01% w/v to about 0.5% w/v, between about 0.025% w/v to about 0.25% w/v, or between about 0.1% w/v to about 0.3% w/v based on the total volume of the aqueous solution. In some embodiments, the concentration of carrageenan in the aqueous solution is about 0.01%, 0.015%, 0.02%, 0.025%, 0.05%, 0.1%, 0.15% 0.2%, 0.25%, 0.3%, 0.35%, 0.4%, 0.45%, or 0.5% w/v based on the total volume of the aqueous solution. According to at least some embodiments or examples described herein, it has been surprisingly identified that an aqueous solution/hydrocolloid comprising between about 0.025% w/v to about 0.25% w/v carrageenan stimulates efficient encystment of T. rostrata, and theronts can be effectively excysted therefrom and infect slugs resulting in decreased grazing and significant mortality.

The concentration of carrageenan in the aqueous solution may also be provided in mg/mL based on the total volume of the aqueous solution. In some embodiments, the concentration of carrageenan in the aqueous solution (in mg/mL) is at least about 0.001, 0.002, 0.005, 0.01, 0.02, 0.05, 0.1, 0.125, 0.15, 0.175, 0.2, 0.25, 0.5, 0.75, 1, 1.25, 1.5, 1.75, 2, 2.25, 2.5, 2.75, 3, 3.5, 4, 4.5 or 5. In some embodiments, the concentration of carrageenan in the aqueous solution (in mg/ml) may be less than about 5, 4.5, 4, 3.5, 3, 2.75, 2.5, 2.25, 2, 1.75, 1.5, 1.25, 1, 0.75, 0.5, 0.25, 0.2, 0.175, 0.15, 0.125, 0.1, 0.05, 0.02, 0.01, 0.005, 0.002, 0.001. Combinations of any two of these upper or lower values to form a range are also possible, for example the concentration of the carrageenan in the aqueous solution is between about 0.002 mg/mL to about 2.5 mg/mL.

In one embodiment, the polymer is gellan gum. In some embodiments, the concentration of gellan gum in the aqueous solution is between about 0.01% w/v to about 20% w/v based on the total volume of the aqueous solution. In some embodiments, the concentration of gellan gum in the aqueous solution is at least about 0.01%, 0.015%, 0.02%, 0.025%, 0.5%, 0.1%, 0.15%, 0.2%, 0.3%, 0.4% 0.5%, 0.6% 0.7%, 0.8%. 0.9%, 1%, 1.1%, 1.2%, 1.3%, 1.4%, 1.5%, 2%, 2.5%, 3%, 3.5%, 4%, 4.5%, 5%, 10%, 15%, or 20% w/v based on the total volume of the aqueous solution. In other embodiments, the concentration of gellan gum in the aqueous solution is less than about 20%, 15%, 10%, 5%, 4.5%, 4%. 3.5%, 3%, 2.5%, 2%, 1.5%, 1.4%, 1.3%, 1.2%, 1.1%, 1%, 0.9%, 0.8%, 0.7%, 0.6%, 0.5%, 0.4%, 0.3%, 0.2%, 0.15%, 0.1%, 0.05%, 0.025%, 0.02%, 0.015%, or 0.01% w/v based on the total volume of the aqueous solution. Combinations of any two of these upper or lower values to form a range are also possible, for example the concentration of gellan gum in the aqueous solution is between about 0.01% w/v to about 15% w/v, between about 0.01% w/v to about 10% w/v, 0.01% w/v to about 5% w/v, or between about 0.01% w/v to about 1% w/v based on the total volume of the aqueous solution.

In some embodiments, the concentration of gellan gum in the aqueous solution is least about 0.01%, 0.015%, 0.02%, 0.025%, 0.05%, 0.1%, 0.15% 0.2%, 0.25%, 0.3%, 0.35%, 0.4%, 0.45%, 0.5%, 1% or 5% w/v based on the total volume of the aqueous solution. In other embodiments, the concentration of gellan gum in the aqueous solution is less than about 5%, 1%, 0.5%, 0.5%, 0.45%, 0.4%, 0.35%, 0.3%, 0.25%, 0.2%, 0.15%, 0.1%, 0.05%, 0.025%, 0.02%, 0.015%, or 0.01% w/v based on the total volume of the aqueous solution. Combinations of any two of these upper or lower values to form a range are also possible, for example the concentration of gellan gum in the aqueous solution is between about 0.01% w/v to about 0.5% w/v, between about 0.025% w/v to about 0.25% w/v, or between about 0.1% w/v to about 0.3% w/v based on the total volume of the aqueous solution. In some embodiments, the concentration of gellan gum in the aqueous solution is about 0.01%, 0.015%, 0.02%, 0.025%, 0.05%, 0.1%, 0.15% 0.2%, 0.25%, 0.3%, 0.35%, 0.4%, 0.45%, or 0.5% w/v based on the total volume of the aqueous solution.

The concentration of gellan gum in the aqueous solution may also be provided in mg/mL based on the total volume of the aqueous solution. In some embodiments, the concentration of gellan gum in the aqueous solution (in mg/mL) is at least about 0.001, 0.002, 0.005, 0.01, 0.02, 0.05, 0.1, 0.125, 0.15, 0.175, 0.2, 0.25, 0.5, 0.75, 1, 1.25, 1.5, 1.75, 2, 2.25, 2.5, 2.75, 3, 3.5, 4, 4.5 or 5. In some embodiments, the concentration of gellan gum in the aqueous solution (in mg/ml) may be less than about 5, 4.5, 4, 3.5, 3, 2.75, 2.5, 2.25, 2, 1.75, 1.5, 1.25, 1, 0.75, 0.5, 0.25, 0.2, 0.175, 0.15, 0.125, 0.1, 0.05, 0.02, 0.01, 0.005, 0.002, 0.001. Combinations of any two of these upper or lower values to form a range are also possible, for example the concentration of gellan gum in the aqueous solution is between about 0.1 mg/mL to about 2.5 mg/mL.

In one embodiment, the polymer is xanthan gum. In some embodiments, the concentration of xanthan gum in the aqueous solution is between about 0.01% w/v to about 20% w/v based on the total volume of the aqueous solution. In some embodiments, the concentration of xanthan gum in the aqueous solution is at least about 0.01%, 0.015%, 0.02%, 0.025%, 0.5%, 0.1%, 0.15%, 0.2%, 0.3%, 0.4% 0.5%, 0.6% 0.7%, 0.8%. 0.9%, 1%, 1.1%, 1.2%, 1.3%, 1.4%, 1.5%, 2%, 2.5%, 3%, 3.5%, 4%, 4.5%, 5%, 10%, 15%, or 20% w/v based on the total volume of the aqueous solution. In other embodiments, the concentration of xanthan gum in the aqueous solution is less than about 20%, 15%, 10%, 5%, 4.5%, 4%. 3.5%, 3%, 2.5%, 2%, 1.5%, 1.4%, 1.3%, 1.2%, 1.1%, 1%, 0.9%, 0.8%, 0.7%, 0.6%, 0.5%, 0.4%, 0.3%, 0.2%, 0.15%, 0.1%, 0.05%, 0.025%, 0.02%, 0.015%, or 0.01% w/v based on the total volume of the aqueous solution. Combinations of any two of these upper or lower values to form a range are also possible, for example the concentration of xanthan gum in the aqueous solution is between about 0.01% w/v to about 15% w/v, between about 0.01% w/v to about 10% w/v, between about 0.01% w/v to about 5% w/v, or between about 0.01% w/v to about 1% w/v based on the total volume of the aqueous solution.

In some embodiments, the concentration of xanthan gum in the aqueous solution is least about 0.01%, 0.015%, 0.02%, 0.025%, 0.05%, 0.1%, 0.15% 0.2%, 0.25%, 0.3%, 0.35%, 0.4%, 0.45%, 0.5%, 1% or 5% w/v based on the total volume of the aqueous solution. In other embodiments, the concentration of xanthan gum in the aqueous solution is less than about 5%, 1%, 0.5%, 0.5%, 0.45%, 0.4%, 0.35%, 0.3%, 0.25%, 0.2%, 0.15%, 0.1%, 0.05%, 0.025%, 0.02%, 0.015%, or 0.01% w/v based on the total volume of the aqueous solution. Combinations of any two of these upper or lower values to form a range are also possible, for example the concentration of xanthan gum in the aqueous solution is between about 0.01% w/v to about 0.5% w/v, between about 0.025% w/v to about 0.25% w/v, or between about 0.1% w/v to about 0.3% w/v based on the total volume of the aqueous solution. In some embodiments, the concentration of xanthan gum in the aqueous solution is about 0.01%, 0.015%, 0.02%, 0.025%, 0.05%, 0.1%, 0.15% 0.2%, 0.25%, 0.3%, 0.35%, 0.4%, 0.45%, or 0.5% w/v based on the total volume of the aqueous solution.

The concentration of xanthan gum in the aqueous solution may also be provided in mg/mL based on the total volume of the aqueous solution. In some embodiments, the concentration of xanthan gum in the aqueous solution (in mg/mL) is at least about 0.001, 0.002, 0.005, 0.01, 0.02, 0.05, 0.1, 0.125, 0.15, 0.175, 0.2, 0.25, 0.5, 0.75, 1, 1.25, 1.5, 1.75, 2, 2.25, 2.5, 2.75, 3, 3.5, 4, 4.5 or 5. In some embodiments, the concentration of xanthan gum in the aqueous solution (in mg/ml) may be less than about 5, 4.5, 4, 3.5, 3, 2.75, 2.5, 2.25, 2, 1.75, 1.5, 1.25, 1, 0.75, 0.5, 0.25, 0.2, 0.175, 0.15, 0.125, 0.1, 0.05, 0.02, 0.01, 0.005, 0.002, 0.001. Combinations of any two of these upper or lower values to form a range are also possible, for example the concentration of xanthan gum in the aqueous solution is between about 0.1 mg/mL to about 2.5 mg/mL.

The aqueous solution comprising the polymer may have a viscosity. The viscosity of the aqueous solution when the polymer is dispersed therein may be higher (e.g. “thicker”) than the native aqueous solution (e.g. no polymer dispersed therein). In some embodiments, the viscosity of the aqueous solution comprising the polymer may be between about 5 to about 1,000 mPa·s when measured using a rotating cylinder method.

In some embodiments, the density of the ciliate cells in the aqueous solution is between about 1×102 cells/mL to about 1×1010 cells/mL. The density of the ciliate cells in the aqueous solution may be at least about 1×102 cells/mL, 1×103 cells/mL, 1×104 cells/mL, 1×105 cells/mL, 1×106 cells/mL, 1×107 cells/mL, 1×108 cells/mL, 1×109 cells/mL, or 1×1010 cells/mL. The density of the ciliate cells in the aqueous solution may be less than about 1×1010 cells/mL, 1×109 cells/mL, 1×108 cells/mL, 1×107 cells/mL, 1×106 cells/mL, 1×105 cells/mL, 1×104 cells/mL, 1×103 cells/mL, or 1×102 cells/mL. Combinations of any two of these upper or lower values to form a range are also possible, for example between about 1×103 cells/mL to about 1×106 cells/mL, between about 1×103 cells/mL to about 1×105 cells/mL, or between about 1×104 cells/mL to about 1×105 cells/mL.

In some embodiments, the aqueous solution comprises at least about 0.5, 1, 2, 5, 10, 15, 20, 25, 30, 35 or 40% w/v ciliate cells. In some embodiments, the aqueous solution comprises less than about 40, 35, 30, 25, 20, 15, 10, 5, 2, 1 or 0.5% w/v ciliate cells. Combinations of these % w/v values are also possible, for example the aqueous solution comprises between about 0.5% w/v to about 40% w/v ciliate cells.

In some embodiments, the vol:vol ratio of the ciliate cells to the aqueous solution is between about 1:10 to about 10:1. In some embodiments, the vol:vol ratio of the ciliate cells to the aqueous solution is at least about 1:10, 1:8, 1:4, 1:2, 1:1, 2:1, 4:1, 6:1, 8:1, or 10:1. In some embodiments, the vol:vol ratio of the ciliate cells to the aqueous solution is less than about 10:1, 8:1, 6:1, 4:1, 2:1, 1:1, 1:2, 1:4, 1:6, 1:8, or 1:10. Combinations of any two of these upper or lower values to form a range are also possible.

It will be appreciated that the embodiments described above and herein in relation to the aqueous solution apply equally to the aqueous solution of the encystment methods, stabilisation methods, and encystment media composition as described herein.

### Additives

The composition may further comprise additional additives, including those described under the heading “Additives” in relation to the method of inducing encystment of ciliate cells. Other examples of additives include preservatives such as parabens, benzoates, sorbic acid, citrates or parabens, humectants such as glycerol or propylene glycol, antioxidants such as butylhydroxytoluene or butylhydroxyanisole, tocopherol, ascorbic acid, flavourings or other formulation auxiliaries. These may be dissolved/suspended in the aqueous solution with the polymer and ciliate cells, or mixed as separate components with the polymer encapsulating the ciliate cells.

In one embodiment, the composition further comprises an attractant or feeding stimulant. As used herein, the term “attractant” refers to an agent which assists in attracting one or more pest species to consume the polymer. As used herein, the term “feeding stimulant” refers to an agent that encourages one or more pest species to remain consuming the polymer for a period of time to allow for the release of ciliate cells suspended or interspersed therein and the pest species to be exposed to the ciliate cells. However, it will be appreciated that the polymers can be consumed by a pest species without the use of attractants and/or feeding stimulants. The attractant may include a pheromone or a nutrient source. In some embodiments, the attractant may be selected from one or more of a starch, carbohydrate, protein, amino acid, plant extracts (e.g. any one of essential oils, saps, resins, chlorophyll and other crude extracts of a plant that a pest species (e.g. slug or snail) may detect as a food source) or a pheromone.

In one embodiment, the composition further comprises a nutrient source. The nutrient source may be a molasses or a sugar. In some embodiments, the nutrient source may be selected from the group consisting of starch, sugar, semolina, couscous or combination thereof. The nutrient source may also be a carbohydrate or a plant product.

In one embodiment, the composition further comprises a feeding stimulant. In some embodiments, the feeding stimulant may be selected from one or more of a starch, carbohydrate, protein, amino acid, plant extracts (e.g. any one of essential oils, saps, resins, chlorophyll and other crude extracts of a plant that a pest species (e.g. slug or snail) may detect as a food source).

In some embodiments, the composition may comprise a single species of ciliate, multiple species of ciliate, or one or more species of ciliates with other organisms, such as pathogenic bacteria, fungal spores, or pathogenic nematodes. Accordingly, in some embodiments, the composition may further comprise one or more other additional components such as one or more of a bait, pesticide, biocontrol agent, or other organisms such as pathogenic bacteria, fungal spores or pathogenic nematodes. In another embodiment, the composition may further comprise one or more metallic salts, for example metallic phosphates and metallic sulfates. In some embodiments, the composition may further comprise iron(III) phosphate (FePO4), iron (II) phosphate (Fe3(PO4)2) and copper (II) sulfate (CuSO4).

In one embodiment, the composition further comprises pathogenic bacteria or fungal spores. The pathogenic bacteria or fungal spores may be suspended within the polymer with the ciliate cells, or may be inside the ciliate cells prior to suspension/interspersion within the polymer. Without wishing to be bound by theory, it is believed that as some bacteria and fungi are pathogenic to pest species, such as slugs and snails, the presence of both ciliate cells and bacteria or fungal spores may result in a higher killing effect. For example, ciliate cells that have ingested bacteria and/or fungi may be released from the polymer and subsequently enter or be ingested by a pest species (e.g. a slug). Once inside the pest species, the ciliate cells may release the bacteria and/or fungi which also has an adverse effect on the pest species, thus resulting in a higher killing effect.

It will be appreciated that the embodiments described above and herein in relation to the additives apply equally to the additives of the encystment methods, stabilisation methods, and encystment media composition as described herein.

### Encystment Media Compositions

The present inventors have also identified an encystment media composition which can be used to induce encystment of trophont ciliate cells. The encystment media composition comprises an aqueous solution and a polymer as described herein. In one embodiment, the encystment media composition comprises an aqueous solution and a non-ionically crosslinked polymer as described herein.

In one embodiment, the aqueous solution comprising the polymer forms a hydrocolloid. In one embodiment, the aqueous solution comprising the polymer forms an anionic hydrocolloid. In one embodiment, the polymer comprises a polysaccharide. In one embodiment, the polysaccharide is an anionic polysaccharide. In one embodiment, the polysaccharide is derived from one or more of algae, plants, animals, fungi, or bacteria. In one embodiment, the polysaccharide is derived from algae. In one embodiment, the algal polysaccharide is selected from the group consisting of carrageenan, agarose, agar and alginate, or a mixture thereof. In one embodiment, the polysaccharide is carrageenan.

In one embodiment, aqueous solution comprises between about 0.1% w/v to about 5% w/v polymer based on the total volume of the aqueous solution. In one embodiment, the aqueous solution comprises between about 0.1% w/v to about 1% w/v polymer based on the total volume of the aqueous solution.

In one embodiment, the aqueous solution is a buffer solution. In one embodiment, the buffer solution comprises a HEPES buffering agent. In one embodiment, the concentration of the HEPES buffering agent in the buffer solution is between about 1 mM to about 25 mM, or between about 5 mM to about 25 mM. In one embodiment, the buffer solution has a pH of between about 6.0 to about 9.0.

In one embodiment, the aqueous solution comprises magnesium ions in an amount effective to stimulate encystment of one or more trophont ciliate cells. In this embodiment, it will be appreciated that concentration of the magnesium ions in the aqueous solution is in a non-crosslinking amount, i.e. the magnesium ions are not present in an amount effective to ionically cross-link the polymer (e.g. carrageenan, xanthan gum and/or gellan gum). In some embodiments, the concentration of the magnesium ions in the aqueous solution is between about 15 μM to about 500 μM. In some embodiments, the concentration of the magnesium ions in the aqueous solution is at least about 15, 20, 30, 50, 100, 150, 200, 250, 300, 350, 400, 450, 500 or 1000 μM. In other embodiments, the concentration of the magnesium ions in the aqueous solution is less than about 1000, 500, 450, 400, 350, 300, 250, 200, 100, 50, 30, 20, or 15 μM. Combinations of these concentration values to form various ranges are also possible, for example the concentration of the magnesium ions in the aqueous solution is between about 15 μM to about 1000 μM, between about 20 μM to about 300 μM, between about 30 μM to about 200 μM, or between about 50 μM to about 150 μM. In one embodiment, the concentration of magnesium ions in the aqueous solution is between about 60 μM to about 65 μM, for example about 62.5 μM. In one embodiment, the aqueous solution comprises magnesium sulfate (MgSO4) or magnesium carbonate (MgCO3). In one preferred embodiment, the aqueous solution comprises magnesium sulfate (MgSO4). It will be appreciated that when the magnesium sulfate or magnesium carbonate is dissolved in the aqueous solution, magnesium ions (Mg2+) are present.

In one embodiment, there is provided a use of an encystment media composition as described herein, for inducing encystment of ciliate cells. In another embodiment, there is provided a method of inducing encystment of ciliate cells, the method comprising incubating a population of trophont ciliate cells in an encystment media composition as described herein at a temperature and for a period of time effective to induce the encystment of the trophont ciliate cells to form one or more encysted ciliate cells.

In addition to the above embodiments, it will be appreciated that the embodiments described above and herein for the polymer, polysaccharide, hydrocolloid, additives and aqueous solution in relation to the composition comprising encysted ciliate cells equally apply to the polymer, polysaccharide, hydrocolloid, additives and aqueous solution of the encystment media composition described herein.

### Method of Inducing Encystment of Ciliate Cells

The present inventors have identified a method of inducing encystment of trophont ciliate cells into encysted ciliate cells using an aqueous solution comprising a polymer, which may be buffered (e.g. with HEPES) to form a buffered aqueous polymer solution. The aqueous solution comprises a polymer as described herein. The aqueous solution comprising the polymer may also be called an aqueous polymer solution. By incubating trophont ciliate cells in the aqueous solution, one or more trophont ciliate cells undergo encystment to form encysted ciliate cells.

In one aspect or embodiment, there is provided a method of inducing the encystment of ciliate cells, the method comprising incubating a population of trophont ciliate cells in an aqueous solution comprising a polymer at a temperature and for a period of time effective to induce the encystment of the trophont ciliate cells to form one or more encysted ciliate cells.

The aqueous solution may be an encystment media composition as described herein. In one embodiment, the aqueous solution comprising the polymer forms a hydrocolloid as described herein. In one embodiment, the polymer is not ionically crosslinked as described herein. In one embodiment, the polymer is not ionically crosslinked during encystment of the trophont ciliate cells.

In one embodiment, the polymer comprises a polysaccharide. In one embodiment, the polysaccharide is an anionic polysaccharide. In one embodiment, the polysaccharide is derived from one or more of algae, plants, animals, fungi, or bacteria. In one embodiment, the polysaccharide is derived from algae or bacteria. In one embodiment, the polysaccharide is selected from the group consisting of carrageenan, xanthan gum, gellan gum, agarose, agar and alginate, or a mixture thereof. In one embodiment, the polysaccharide is carrageenan, xanthan gum or gellan gum.

In one embodiment, aqueous solution comprises between about 0.1% w/v to about 5% w/v polymer based on the total volume of the aqueous solution. In one embodiment, the aqueous solution comprises between about 0.1% w/v to about 1% w/v polymer based on the total volume of the aqueous solution.

In one embodiment, the aqueous solution is a buffer solution. In one embodiment, the buffer solution comprises a HEPES buffering agent. In one embodiment, the concentration of the HEPES buffering agent in the buffer solution is between about 5 mM to about 25 mM. In one embodiment, the buffer solution has a pH of between about 6.0 to about 9.0.

In one embodiment, the method does not comprise adding an effective amount of an ionic cross-linker to the aqueous solution, for example a multivalent metal cation or multivalent anion, in an amount effective to ionically crosslink the polymer to form a hydrogel (e.g. capable of crosslinking the polymer to form a hydrogel as defined above).

In one embodiment, the aqueous solution comprising the polymer is heat-treated prior to incubation with the trophont ciliate cells. In some embodiments, the aqueous solution is heated to a temperature (in ° C.) of at least about 30, 40, 50, 60, 60, 70, 80, 90, 100, 110, 120 or 130 prior to incubation with the trophont ciliate cells. In some embodiments, the aqueous solution is heated to a temperature (in ° C.) of less than about 130, 120, 110, 100, 90, 80, 70, 60, 50, 40 or 30 prior to incubation with the trophont ciliate cells. The heating temperature may be in a range provided by any two of these upper and/or lower values, for example between about 90° C. to about 130° C. The heat treatment may be performed by any suitable method, for example heating the aqueous solution on a hotplate or in an autoclave. In one embodiment, the aqueous solution is heat-treated for a period of time (in minutes) or at least 1, 2, 5, 10, 15, 20, 25, 30 or 60 prior to incubation with the trophont ciliate cells. The heat treatment period may be a range provided by any two of these upper and/or lower values, for example between about 5 minutes to about 20 minutes. Following heating, the aqueous solution may be cooled to room temperature prior to incubation with the trophont ciliate cells. In an alternative embodiment, the aqueous solution comprising the polymer is not heat-treated prior to incubation with the trophont ciliate cells. According to some embodiments or examples described herein, the presence or absence of a heat treatment step prior to incubation may improve encystment efficiency due to altering the internal hydrocolloid structure. Nonetheless, the compositions of the present invention induced encystment of trophont ciliate cells irrespective of whether the aqueous solution was heat treated prior to incubation, highlighting the versatility of the encystment composition described herein. In one embodiment, the method comprises including

The embodiments described above and herein in for the ciliate cells, polymer, polysaccharide, hydrocolloid, aqueous solution and additives in relation to the composition comprising encysted ciliate cells and encystment media equally apply to the ciliate cells, polymer, polysaccharide, hydrocolloid, aqueous solution and additives of the method of inducing encystment of ciliate cells, where applicable.

Further additional embodiments specific to the method of inducing encystment are described below.

### Incubation, Storage and Dehydration

In some embodiments, the trophont ciliate cells are young trophont ciliate cultures, for example have undergone less than 10, 9, 8, 7, 6, 5, 4, 3, 2 or 1 subcultures. The number of subcultures give an indication of the number of cell generations that have occurred since cells last went through autogamy. Trophont ciliate cultures that have undergone less than 10 subcultures are considered to contain “young” trophont cells.

In some embodiments, the vol:vol ratio of the population of ciliate cells to the aqueous solution is between about 1:10 to about 10:1. In some embodiments, the vol:vol ratio of the population of ciliate cells to the aqueous solution is at least about 1:10, 1:8, 1:4, 1:2, 1:1, 2:1, 4:1, 6:1, 8:1, or 10:1. In some embodiments, the vol:vol ratio of the population of ciliate cells to the aqueous solution is less than about 10:1, 8:1, 6:1, 4:1, 2:1, 1:1, 1:2, 1:4, 1:6, 1:8, or 1:10. Combinations of any two of these upper or lower values to form a range are also possible.

The population of trophont ciliate cells are incubated in an aqueous solution comprising a polymer at a temperature and for a period of time effective to induce the encystment of the trophont ciliate cells to form one or more encysted ciliate cells.

In some embodiments, the trophont ciliate cells may be incubated in the aqueous solution at a temperature effective to induce encystment to form encysted ciliate cells. The temperature may be between 10° C. to about 40° C. The temperature may be at least about 10, 15, 20, 25, 30, 35 or 40° C. The temperature may be less than about 40, 35, 30, 25, 20, 15 or 10° C. Combinations of any two of these upper or lower values to form a range is possible, for example between about 20° C. to about 30° C.

In some embodiments, the trophont ciliate cells may be incubated in the aqueous solution at a temperature of at least about 20, 21, 22, 23, 24, 25, 26, 27, 28, 29 or 30° C. The temperature may be less than about 30, 29, 28, 27, 26, 25, 24, 23, 22, 21 or 20° C. Combinations of any two of these upper or lower values to form a range is possible, for example between about 20° C. to about 28° C. In some embodiments, the trophont ciliate cells may be incubated in the aqueous solution at a temperature of about 20, 21, 22, 23, 24, 25, 26, 27, 28, 29 or 30° C.

In some embodiments, the trophont ciliate cells may be incubated in the aqueous solution for a period of time effective to induce encystment to form encysted ciliate cells. The period of time may be between about 0.5 hours to about 96 hours. The period of time may be at least about 12, 24, 36, 48, 60, 72, 84, or 96 hours. The period of time may be less than about 96, 84, 72, 60, 48, 36, 24 or 12 hours. Combinations of any two of these upper or lower values to form a range is possible, for example between about 12 hours to about 72 hours.

In some embodiments, the trophont ciliate cells may be incubated in the aqueous solution at a temperature and for a period of time effective to induce encystment to form encysted ciliate cells (i.e. an encystment temperature) and then subsequently incubated at a temperature effective to store the encysted ciliate cells (i.e. a storage temperature). It will be appreciated that the encystment and storage temperature may be the same. For example, the ciliate cells may be incubated at i) a first temperature to induce encystment to form encysted ciliate cells and then ii) transferred to a second temperature to store the encysted ciliate cells.

The encystment temperature i) may be at least about 20, 21, 22, 23, 24, 25, 26, 27, 28, 29 or 30° C. The encystment temperature may be less than about 30, 29, 28, 27, 26, 25, 24, 23, 22, 21 or 20° C. Combinations of any two of these upper or lower values to form a range is possible, for example between about 10° C. to about 30° C., e.g. about 26° C. The storage temperature ii) may be at least about 15, 16, 17, 18, 19, 20, 21, 22, 23, 24, 25, 26, 27, 28, 29 or 30° C. The storage temperature may be less than about 30, 29, 28, 27, 26, 25, 24, 23, 22, 21, 20, 19, 18, 17, 16, or 15° C. Combinations of any two of these upper or lower values to form a range is possible, for example between about 10° C. to about 30° C., or between about 15° C. to about 25° C., e.g. about 20° C.

The encystment temperature i) may be maintained for a period of time of between about 0.5 hours to about 96 hours. The encystment temperature may be maintained for at least about 12, 24, 36, 48, 60, 72, 84, or 96 hours. The encystment temperature may be maintained for less than about 96, 84, 72, 60, 48, 36, 24 or 12 hours. Combinations of any two of these upper or lower values to form a range is possible, for example between about 12 hours to about 72 hours. The storage temperature ii) may be maintained for a period of time of at least 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 12 weeks, 15 weeks, or 20 weeks.

The method may further comprise the step of dehydrating the aqueous solution to obtain a dried polymer wherein the one or more encysted ciliate cells are interspersed within the polymer. The dehydration step essentially evaporates the aqueous solution leaving a dried polymer. It will be appreciated that the polymer can be rehydrated upon contact with a suitable aqueous environment, such as water (e.g. rain or a sprinkler following application to an environment) or an aqueous solution as described herein. The aqueous solution may be dried using any conventional means, such as room temperature airflow, mild heat and/or vacuum. The ciliate cells interspersed within the dried polymer remain viable even after dehydration. Encystment of trophonts to ciliate cells may occur during dehydration.

The aqueous solution may be dehydrated from a humidity under ambient conditions (e.g. an ambient humidity at 20° C. and atmospheric pressure) to a reduced humidity. For example, dehydrating the aqueous solution will result in a relative humidity of less than 100%. In some embodiments, the aqueous soil solution comprising the incubated trophont ciliate cells and suspended soil particles is dehydrated to a relative humidity of less than about 100%, 95%, 90%, 85%, 80%, 75%, 70%, 65%, 60%, 55%, 50%, 45%, or 40%, for example less than about 80%, 75%, or 70%. Combinations of these relative humidities are also possible, for example about 40% to about 75% relative to the ambient humidity. The dehydrated environment may be obtained by any suitable means, including for example using humidity chambers. The humidity levels may be measured by any routine means including a humidity monitor or hygrometer, such as a gravimetric hygrometer. In some embodiments, the aqueous solution is dehydrated for at least about 0.5, 1, 2, 3, 4, 5, 8, 10, 12, 15, 18, 20, 24 or 30 days. In some embodiments, the aqueous solution is dehydrated at a temperature of between about 20° C. to about 30° C., for example about 20° C.

In some embodiments, the hydrocolloid comprising the ciliate cells may be dried (e.g. dehydrated) to form a dried polymer/hydrocolloid comprising the ciliate cells.

In some embodiments, prior to incubating in the aqueous polymer solutions, the trophont cells were obtained via culturing T. rostrata in PPYE media (0.5% (w/v) proteose peptone (Oxoid LP0085), 0.5% (w/v) yeast extract (Oxoid LP0021), and 0.125% (w/v) glucose) or PP media (1% w/v Proteose Peptone (Oxoid LP0085) and 0.125% w/v glucose). In one embodiment, the trophont cells are then subcultured in PP media. Using trophont cells cultured in PPYE and sub cultured in PP media may provide further advantages such as increased cyst resilience following encystment.

In some embodiments, the encysted ciliate cells are subsequently transferred to fresh nutrient medium where they undergo excystment to form theront ciliate cells. Alternatively, the encysted ciliate cells may be stabilised and stored at a suitable temperature and for a period of time as described herein, for example at 20° C. or by dehydration.

### Additives

One or more additives may be added to the aqueous solution either prior to or during incubation (e.g. prior to or during) encystment or after incubation (e.g. after encystment) of the ciliate cells.

In one embodiment, magnesium ions may be added to the aqueous solution prior to or during incubation in an amount effective to stimulate encystment of one or more trophont ciliate cells. In this embodiment, it will be appreciated that concentration of the magnesium ions added to the aqueous solution prior to or during incubation is in a non-crosslinking amount, i.e. the magnesium ions are not present in an amount effective to ionically cross-link the polymer (e.g. carrageenan, xanthan gum and/or gellan gum) in which the trophont ciliate cells are interspersed therein.

In some embodiments, the concentration of the magnesium ions in the aqueous solution prior to or during incubation is provided in an amount effective to stimulate encystment of the one or more trophont ciliate cells, for example between is about 15 μM to about 500 μM. In some embodiments, the concentration of the magnesium ions in the aqueous solution prior to or during incubation is at least about 15, 20, 30, 50, 100, 150, 200, 250, 300, 350, 400, 450, 500 or 1000 μM. In other embodiments, the concentration of the magnesium ions in the aqueous solution prior to or during incubation is less than about 1000, 500, 450, 400, 350, 300, 250, 200, 100, 50, 30, 20, or 15 μM. Combinations of these concentration values to form various ranges are also possible, for example the concentration of the magnesium ions in the aqueous solution prior to or during incubation is between about 15 μM to about 1000 μM, between about 20 μM to about 300 μM, between about 30 μM to about 200 μM, or between about 50 μM to about 150 μM. In one embodiment, the concentration of magnesium ions in the aqueous solution prior to or during incubation is between about 60 μM to about 65 μM, for example about 62.5 μM. The magnesium ions (e.g. provided by magnesium sulfate) may be added to the aqueous solution at any point prior to or during incubation to stimulate encystment of one or more trophont ciliate cells.

Alternatively or additionally, the method may further comprise the step of adding magnesium ions to the aqueous solution following incubation (e.g. following encystment) in an amount effective to stabilise the one or more encysted ciliate cells. According to some embodiments or examples described herein, the present inventors have found that adding magnesium ions to the buffer solutions comprising encysted ciliate cells prior to storing stabilised the one or more encysted cells for a prolonged period of time.

In some embodiments, the concentration of magnesium ions added to the aqueous solution following incubation is in an amount effective to stabilise encysted ciliate cells, for example between about 25 mM to about 100 mM. In some embodiments, the concentration of magnesium ions added to the aqueous solution following incubation is at least about 25, 30, 35, 40, 45, 50, 55, 60, 65, 70, 75, 80, 90 or 100 mM. In other embodiments, the concentration of magnesium ions added to the aqueous solution following incubation is less than about 100, 90, 80, 75, 70, 65, 60, 55, 50, 40, 35, 30 or 25 mM. Combinations of these concentration values to form various ranges are also possible, for example the concentration of magnesium ions added to the aqueous solution following incubation is between about 25 mM to about 75 mM, for example about 50 mM. In one embodiment, the ciliate cells are incubated with the aqueous solution for at least about 6, 12, 18 or 24 hours prior to the addition of magnesium ions. In one embodiment, the method further comprises the step of adding magnesium ions to the aqueous solution following encystment to stabilise the one or more encysted ciliate cells. In one embodiment, the magnesium ions are added to the aqueous solution after incubation but prior to storing the encysted ciliate cells. In one embodiment, the magnesium ions are added to the aqueous solution after incubation but prior to transferring the aqueous solution from the encystment temperature to the storage temperature. According to some embodiments or examples, the addition of magnesium ions may stabilise encysted cells at lower polymer concentrations.

The magnesium ions described herein may be provided by a suitable magnesium salt, for example magnesium sulfate (MgSO4) or magnesium carbonate (MgCO3). In one embodiment, the magnesium ions are provided by magnesium sulfate. It will be appreciated that when the magnesium sulfate or magnesium carbonate is dissolved in the aqueous solution, magnesium ions (Mg2+) are present.

The embodiments described above and herein in relation to the magnesium ions as an additive in relation to the method of inducting encystment equally apply to magnesium ions present as an additive in the compositions described herein.

In some embodiments, one or more bacterial metabolites, including for example one or more bacterial polysaccharides as described herein, may be added to the aqueous solution. The bacterial metabolites may be derived from composted pine bark particles.

The composted pine bark particles may have an average particle size of between about 1 μm to about 200 μm. The composted pine bark particles may have an average particle size of at least about 1, 5, 10, 15, 20, 25, 30, 40, 50, 60, 80, 100, or 120 μm. The composted pine bark particles may have an average particle size of less than about 120, 100, 80, 60, 50, 40, 30, 25, 20, 15, 10, 5 or 1 μm. Combinations of average particle sizes are also possible, for example the composted pine bark particles may have an average particles size of between about 5 μm to about 100 μm, or between about 5 μm to about 60 μm. The composted pine bark particles may have an average particle size of less than about 60 μm. In one embodiment, the composted pine bark particles may have an average particle size of less than about 30 μm. As used herein, the term “average particle size” refers to a mean average particle size as determined by sieve analysis.

In some embodiments, the aqueous solution comprises between about 0.01% w/v to about 10% w/v composted pine bark particles based on the total volume of the aqueous solution. In some embodiments, the aqueous solution comprises at least about 0.001, 0.01, 0.02, 0.05, 0.08, 0.1, 0.2, 0.5, 1, 2, 5, or 10% w/v composted pine bark particles based on the total volume of the aqueous solution. In some embodiments, the aqueous solution comprises less than 10, 5, 2, 1, 0.5, 0.2, 0.1, 0.08, 0.05, 0.02, 0.01 or 0.001% w/v composted pine bark particles based on the total volume of the aqueous solution. Combinations of these ranges are also possible.

Alternatively or additionally, the aqueous solution may comprise other particles, such as algal cells, starch (e.g. corn starch), grains (e.g. rice), activated charcoal, magnesium silicate, polystyrene and dextrans (e.g. sulphopropyl and quaternary ammonia ethyl substituted dextrans) or bacterial cells, including in the % w/v amounts recited above for the composted pine bark particles.

The aqueous solution and compositions described herein may comprise any one or more additives as described above and herein in relation to the method of encysting ciliate cells.

### Method of Stabilising Ciliate Cells

The polymer solutions described herein can also stabilised ciliate cells.

In one aspect or embodiment, there is provided a method of stabilising encysted ciliate cells, the method comprising suspending a population of encysted ciliate cells in an aqueous solution comprising a polymer as described herein.

The encysted ciliate cells may be obtained by methods described herein, or one or more alternative encystment methods. For example, the encysted ciliate cells may be obtained by incubating a population of trophont ciliate cells in a buffer solution comprising magnesium ions, or by incubating a population of trophont ciliate cells in an aqueous solution comprising suspended soil particles (i.e. an aqueous soil solution), wherein the trophont ciliate cells undergo encystment to form one or more encysted ciliate cells. Alternatively, the encysted ciliate cells are obtained by incubating a population of trophont ciliate cells in an aqueous solution comprising a polymer at a temperature and for a period of time effective to induce the encystment of the trophont ciliate cells to form one or more encysted ciliate cells, as described herein.

In another aspect or embodiment, there is provided a method of stabilising excysted ciliate cells (e.g. theronts), the method comprising suspending a population of excysted ciliate cells in an aqueous solution comprising a polymer as described herein.

It will be appreciated that the embodiments provided above for the polymer, ciliate cells, aqueous solution and additives in relation to the composition, encystment media and method of inducing encystment also equally apply to the polymer, ciliate cells, aqueous solution and additives to the method of stabilising encysted ciliate cells, where applicable.

### Storage, Stability and Viability of Ciliate Cells

The polymers, aqueous solutions and/or hydrocolloids comprising the ciliate cells are stable and the ciliate cells remained viable during storage. In one embodiment, the ciliate cells within the polymer (either as an aqueous solution, hydrocolloid or dried polymer) remained as encysted ciliate cells during storage. For example, the present inventors discovered that, in some embodiments, trophont ciliate cells that were incubated within an aqueous polymer solution underwent encystment to form encysted ciliate cells, and remained as encysted ciliate cells during storage. Additionally, it was also identified that if pre-formed encysted cells were suspended or interspersed within the polymer as described herein, they also remained as encysted ciliate cells during storage. This demonstrates that ciliate cells can be stored and remain stable when suspended in polymers of the present disclosure.

In some embodiments, nearly all of the encysted ciliate cells within the polymer remained as encysted ciliate cells and viable for at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 12 weeks, 15 weeks, 20 weeks, 6 months, 12 months, 18 months or 24 months, highlighting the effect suspension/interspersion within the polymer (e.g. carrageenan) has on ciliate cell stability. In some embodiments, the polymer comprising encysted ciliate cells is stored in a sealed container.

In one embodiment, the ciliate cells suspended or interspersed within the polymer can be stored under ambient conditions (e.g. in the dark, room temperature). For example, the storage temperature may be between about 1° C. to about 30° C. In some embodiments, the storage temperature may be at least about 1, 2, 3, 4, 5, 6, 7, 8, 9, 10, 12, 14, 16, 18, 20, 22, 24, 26, 28, or 30° C. In other embodiments, the storage temperature may be less than about 30, 28, 26, 24, 22, 20, 18, 16, 14, 12, 10, 9, 8, 7, 6, 5, 4, 3, or 2° C. Combinations of these storage temperatures to form various ranges are also possible, for example, between about 3° C. to about 25° C., or between about 4° C. to about 28° C.

For example, the present inventors have identified that, in some embodiments, the encysted ciliate cells remained encysted and viable after 8 weeks storage at 20° C. These encysted cells remained viable and following release from the polymer, the encysted cells were able to excyst into theront cells and establish new populations in culture, highlighting the improved storage properties of the polymers of the present invention.

In another embodiment, the ciliate cells suspended or interspersed within the polymer can be stored in the dark. In some embodiments, the ciliate cells suspended or interspersed within the polymer can be stored with minimal to no additional moisture.

In some embodiments, the suspended or interspersed ciliate cells can subsequently be released from the polymer into the external environment. For example, the polymer can be suspended/soaked in water which softening the polymer structure allowing for the encysted or encapsulated cells to be released to the external environment.

In some embodiments, the encysted cells within the polymer are able to undergo excystment to form theront ciliate cells upon release from the polymer. For example, when the polymer comprising the encysted ciliate cells is suspended in fresh media (e.g. sodium citrate buffer or PPYE medium), the encysted ciliate cells are gradually released which then undergo excystment to form theront ciliate cells. The theront ciliate cells can then mature into a healthy new trophont cell cultures.

Methods and Compositions for Infecting or Colonising a Pest Species with a Ciliate

Theront cells are the infective form of T. rostrata which were more effective at killing or reducing the fitness of slugs faster than trophont ciliate cells. In some embodiments, the compositions of the present invention can be used to transport and deliver viable and stable encysted ciliate cells to a pest species and/or to an area affected or likely to be affected by a pest species (e.g. slugs or snails), where once the cells are released from within the polymer, they can undergo excystment to form the infective theront ciliate cells which are released into the environment and infect the pest species.

In other embodiments, the compositions of present invention can be used to transport and deliver viable and stable excysted ciliate cells (e.g. theronts) to a pest species and/or to an area affected or likely to be affected by a pest species (e.g. slugs or snails). According to some embodiments or examples described herein, it has been identified that the compositions described herein can also stabilise theronts which remain viable and infective for a sufficient period of time, which can then be directly applied to pest species as described herein.

Therefore, in some embodiments, the compositions and ciliate cells as described herein can be dispersed in the environment for infection or colonisation of pests. In one embodiment, the aqueous polymer solutions or polymers suspending ciliate cells as described herein can be dispersed in the environment for infection or colonisation of pests.

In a related aspect or embodiment, there is provided a method of infecting or colonising a pest species with a ciliate, the method comprising applying to an area affected or likely to be affected by a pest species a composition described herein or an encysted ciliate cell prepared or stabilised by the methods described herein, or a composition as described herein. In a related aspect or embodiment, there is provided a method of infecting or colonising a pest species with a ciliate, the method comprising applying a composition as described herein or an encysted ciliate cell prepared or stabilised by the method as herein to the pest species.

As used herein, the term “colonisation” or variants thereof refers to the entry of the protist to an external facing tissue or orifice of the pest such as the sub-mantle tissue lining or gut tissue following ingestion of the polymer or ciliate cells released therefrom. The pest species may consume the polymer and release the encapsulated or suspended ciliate cells.

In some embodiments, the compositions or ciliate cells can be applied to the pest species (e.g. applied directly to the pest species). Such direct application may include spraying the composition or ciliate cells onto the pest species.

In other embodiments, the compositions or ciliate cells can be applied to an agricultural, water (i.e. aquaculture) environment and/or horticultural environment. In one embodiment, the compositions or ciliate cells may be applied to an area of soil affected by a pest (such as a slug or a snail). For example, in relation to the aqueous polymer solution or polymer comprising the ciliate cells may dissolve/disintegrate in the moist soil environment and release the ciliate cells into the soil. The encysted ciliate cells released may subsequently undergo excystment within the soil into theront ciliate cells which are infective and can infect various pest species living in, on or around the soil area. If the cells released from the polymer are trophont ciliate cells, then once released, the trophont ciliate cells may encyst in the soil environment to form encysted ciliate cells, which can then subsequently excyst to form the infective theront ciliate cells, as per the life cycle in FIG. 1 or can invade the pest animal tissue directly. The released ciliate cells may also be ingested by the pest, and undergo transformation within the pest to form theront ciliate cells. In yet another embodiment, if the released ciliate cells are theront ciliate cells, these may go on to infect the pest species.

In other embodiments, the compositions or ciliate cells could be applied to an area and subsequently wetted (e.g. by the rain, sprinkler, inundation, or drip irrigation etc.) wherein the water environment promotes the release of the ciliate cells. For example, a dried polymer (e.g. carrageenan) may dissolve in the moist environment and release the ciliate cells, which can go on to form theront ciliate cells, and subsequently infect pest species. In some embodiments, the compositions or ciliate cells are applied directly to the pest species (e.g. by spraying onto the pest species). It will be appreciated that such direct application may occur simultaneously with the application of the composition or ciliate cells to an area affected or likely to be affected by a pest species as described herein. For example, by directly applying the compositions or ciliate cells to the pest species, the infected pest species may then spread the theronts to niches where other pests take refuge thus promoting colonising and infection.

Other areas that may be affected by pests that the compositions and ciliate cells of the present invention can be applied to include farms, gardens, crops, nurseries, pastures, fields, greenhouses, shadehouses, hydroponic nurseries.

In one embodiment, the area or environment the compositions or ciliate cells are applied to is an agricultural crop. In one aspect or embodiment, there is provided a method for reducing agricultural crop damage comprising applying to an agricultural crop area affected or likely to be affected by a pest species a pest species a composition described herein or an encysted ciliate cell prepared or stabilised by the methods described herein, or a composition as described herein.

In another aspect or embodiment, there is provided an agricultural or horticultural composition comprising the composition described herein or an encysted ciliate cell prepared or stabilised by the methods described herein, in combination with one or more agriculturally or horticulturally acceptable carriers.

In another embodiment, the compositions or ciliate cells may be added to a container with holes to allow access for pest species, for example commercial traps (refuges) currently on the market for pest control, such as slug and snail traps. Without wishing to be bound by theory, placing the compositions or ciliate cells in such traps may expose the pests long enough for them to get infected with the ciliate cells which then kills them, or even if the pests manage to leave the trap, the infected pest would disperse the ciliates as they migrate around the adjacent area, thus spreading the ciliate infection to other pests in the area. The trap can then be refilled with more of the compositions or ciliate cells as the pest species consumes them. Other advantages may be provided by such using traps to deliver the compositions or ciliate cells to pest species, such as portability to different garden areas as damage to plants is observed indicating the presence of pests requiring control. It will be appreciated that other delivery methods are also envisaged and the above examples are not to be considered limiting.

In some embodiments, the compositions or ciliate cells may comprise or be further mixed with one or more acceptable carriers. The acceptable carrier may be an agriculturally or horticulturally acceptable carrier. As used herein, an “acceptable carrier” and/or an “agriculturally acceptable carrier” and/or a “horticulturally acceptable carrier” is any carrier on which can facilitate the transport of the compositions or ciliate cells to an area affected or likely to be affected by a pest species (such as an invertebrate), and which is otherwise suitable for agricultural or horticultural use. Any such suitable acceptable carrier can be used, including but not limited to seeds, seed coats, granular carriers, liquid slurry carriers, and liquid suspension carriers.

Suitable agriculturally or horticulturally acceptable carriers include fillers, solvents, excipients, surfactants, suspending agents, spreaders/stickers (adhesives), antifoaming agents, dispersants, wetting agents, drift reducing agents, auxiliaries, adjuvants or a mixture thereof. For example, the agriculturally or horticulturally acceptable carrier may be selected from the group consisting of a filler stimulant, an anti-caking agent, a wetting agent, an emulsifier, and an antioxidant, for example said composition comprises at least one of each of a filler stimulant, an anti-caking agent, a wetting agent, an emulsifier, and an antioxidant.

In one embodiment solid carriers include but are not limited to mineral earths such as silicic acids, silica gels, silicates, talc, kaolin, attapulgus clay, limestone, lime, chalk, bole, loess, clay, bentonite, dolomite, diatomaceous earth, aluminas calcium sulfate, magnesium sulfate, magnesium oxide, peat, humates, ground plastics, fertilizers such as ammonium sulfate, ammonium phosphate, ammonium nitrate, and ureas, and vegetable products such as grain meals, bark meal, wood meal, and nutshell meal, cellulosic powders, seaweed powders, peat, talc, carbohydrates such as mono-saccharides and di-saccharides, starch extracted from corn or potato or tapioca, chemically or physically altered corn starch and the like. As solid carriers for the compositions, cells and strains of the present invention, the following are suitable as carriers: crushed or fractionated natural rocks such as calcite, marble, pumice, sepiolite and dolomite; synthetic granules of inorganic or organic meals; granules of organic material such as sawdust, coconut shells, corn cobs, corn husks or tobacco stalks; kieselguhr, tricalcium phosphate, powdered cork, or absorbent carbon black; water soluble polymers, resins, waxes; or solid fertilizers. Such solid compositions may, if desired, contain one or more compatible wetting, dispersing, emulsifying or colouring agents which, when solid, may also serve as a diluent.

The acceptable carrier preferably has a sufficient shelf life, and preferably assists in the dispersion of the compositions, ciliate cells and/or isolated strains to an area affected or likely to be affected by a pest species (such as an invertebrate).

The pest species can be an invertebrate or a vertebrate. In one embodiment, the vertebrate is a lower vertebrate.

In one embodiment, the pest species is an invertebrate. The invertebrate may be a mollusc or arthropod, such as a dipteran (e.g. a mosquito). In another embodiment, the pest species is a vertebrate, for example a fish species. In a preferred embodiment, the invertebrate is a mollusc, for example a Gastropod.

The Gastropod may be a snail or a slug. The slugs and snails to be controlled in include all land-dwelling slugs and snails, for example those which occur as polyphagus pests in agricultural and horticultural crops. Agriculturally and horticulturally problematic slug and snail types are, for example, slugs such as the invasive Anion ater group such as A ater, A rufus, and A vulgaris). Other non-limiting examples of slugs to be controlled include Ambigolimax valentianus, Deroceras invadens, Limacus flavus, Deroceras reticulatum (i.e. grey field slugs).

### EXAMPLES

### Example 1—Materials and Methods

All media and solutions were prepared fresh using laboratory grade water and sterilised by autoclaving at 121° Celsius for 20 minutes unless otherwise noted. PPYE media consisted of 0.5% (w/v) proteose peptone (Oxoid LP0085), 0.5% (w/v) yeast extract (Oxoid LP0021), and 0.125% (w/v) glucose. PP medium was PPYE without yeast extract (0.5% w/v Proteose Peptone (Oxoid LP0085) and 0.125% w/v glucose).

Isolation of T. rostrata TRAUS

Strain TRAUS was isolated from an egg laid by a F1 laboratory-reared D. reticulatum whose parents were collected from Melbourne, Australia. The egg was surface sterilised in 0.01% v/v hypochlorite for 5 min then washed several times in sterile distilled water to remove the hypochlorite. The egg was aseptically opened using a needle to release the ciliates into water and then immediately transferred to 10 ml of SPP media in a 25 cm2 tissue culture flask with a vented lid (IWAKI).

### Preparation of 0.25% w/v Carrageenan Buffer Solution (0.25% w/v Carrageenan-H)

To prepare the 0.25% w/v carrageenan buffer solution, 1.5 g carrageenan (Sigma C1013, CAS Number: 9000-07-1) was mixed with 600 mL of water either at 17-20° C. or 90-100° C. for 15 minutes, or autoclaved at 121° C. for 10 minutes, and then allowed to cooled before addition of 10 mM HEPES pH 7 NaOH buffer.

Preparation of 0.125% Carrageenan Buffer Solution (0.125% w/v Carrageenan-H)

To prepare the 0.125% w/v carrageenan buffer solution, 0.75 g carrageenan (Sigma C1013, CAS Number: 9000-07-1) was mixed with 600 mL of water either at 17-20° C. or 90-100° C. for 15 minutes, or autoclaved at 121° C. for 10 minutes, and then allowed to cooled before addition of 10 mM HEPES pH 7 NaOH buffer

### Preparation of Composted Pine Bark Infusion Buffer (CI—H)

A composted pine bark infusion buffer (CI—H) was prepared, according to a modified protocol by Segade et al. (2016) by mixing 50 g of medium grade composted pine bark (Pinus radiata; Australian Growing Solutions, Tyabb, Victoria, Australia) in Milli Q ultrapure water. The suspension was maintained in agitation for 15 min at room temperature (20° C.) and then large particles were removed by sieving and/or centrifugation. The decanted suspension was sterilised by autoclaving and HEPES buffer pH 7 was added to a final concentration of 10 mM.

### Preparation of Filtered Composted Pine Bark Wash (UP6 and UP4)

UP6 and UP4 filtered composted pine bark infusion buffers were prepared by washing the fine surface layer particles out from approximately 500 g of the medium grade composted pine bark, in two batches in a 400 micron sieve (hop spider), with reverse osmosis water. The flow through was then passed through a 50 micron Nitex mesh netting cloth (Australian Entomological Supplies) and the collected filtrate was sterilised by autoclaving, then stored at room temperature.

Harvesting Dried Particles from CI (DCIP), UP4 (DUP4) and UP6 (DUPE)

Particles from the infusions were harvested by centrifugation at 800 g for 10 min. The particles were then air dried, and then rehydrated to the desired concentration.

### Preparation of MgSO4 Buffer Solution

Magnesium sulfate (MgSO4) was dissolved in ultrapure water at a concentration of 62.5 μM and subsequently autoclaved at 121° C. for 20 minutes.

### Encystment of Trophont Ciliate Cells in Carrageenan Buffer Solutions.

T. rostrata trophonts were obtained using an isolated strain of T. rostrata TRAUS (deposited under PTA-126056 on 13 Aug. 2019 at the American Type Culture Collection) and were cultivated at 20° C. in PPYE and subcultured in PP. The density of the culture was assessed by a direct cell counting using a haemocytometer indicating the culture was 4.6×104 cell/ml.

Trophonts grown in PP media were harvested (800×g 10 min), washed in 2 volumes of 10 mM HEPES pH 7 NaOH (HEPES) and resuspended to a volume equivalent to 5×104 original trophonts per ml in either 1) 0.25% carrageenan-H; 2) 0.125% carrageenan-H; or 3) CI—H, and subsequently transferred to tissue culture plates and incubated under various conditions to evaluate the response of trophonts to incubation in the solutions at different temperatures.

Trophonts are tear-drop shaped (rostrate) whereas they change to a rounded form during encystment into encysted ciliate cells. The rounding of the trophonts was the indication that the conditions may stimulate cyst formation. Rounded cells were examined microscopically for characteristic cyst capsules. The viability of the cyst-like cells was assessed by restoring the cells to conditions permissive for growth and measuring the viability as the ability to multiply using the Most Probable Number Method (MPN) over 31 days. T=0 (days) from the commencement of the PP trophont culture. For the viability counts, trophonts were determined as viable through their movement and were directly counted. All other viability counts on the cultures were done by determining MPN on 3 individual cultures and the 95% confidence limits are shown. The percent round cells counts on the incubated cultures were determined from their individual cultures with the maximum and minimum values.

### Lethality Experiments

Laboratory-reared D. reticulatum were maintained as previously described by Billman-Jacobe et al. (2020). Young slugs, ˜0.8 cm long, were selected for all experiments. Each slug was housed in a 25 ml tube containing 3 g of moist potting soil (Plugger 111-Seedraising Mix, Australian Growing Solutions). The tubes were placed in a humidified box and kept in a controlled environment at 17° C. with a 12-hour photoperiod. There were 20 slugs per treatment. The grazing activity and mortality of slugs was assessed daily or weekly, depending on the experiment. Each slug was provided Chinese cabbage for food and the cabbage was replaced as required.

For experiments, 20 replicates of individually-housed D. reticulatum slugs were exposed to 105 or zero trophonts or theronts. The theronts and trophonts were concentrated by centrifugation at 800 g for 10 min for the challenge. Trophonts used for challenge experiments were derived from cysts and cultured in PPYE for 4 days to stimulate excystment and conversion to trophonts. Trophonts were washed free of media using 10 mM HEPES pH 7 NaOH and stored at 20° C. for 1 day at a density of 3×104 cells/ml (a density at which the trophonts do not encyst at 20° C.).

### Example 2: Carrageenan Buffer Solution Induces Encystment of T. rostrata at 26° C.

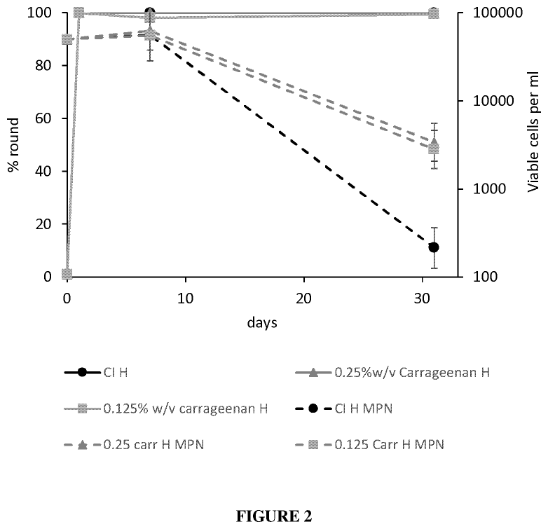

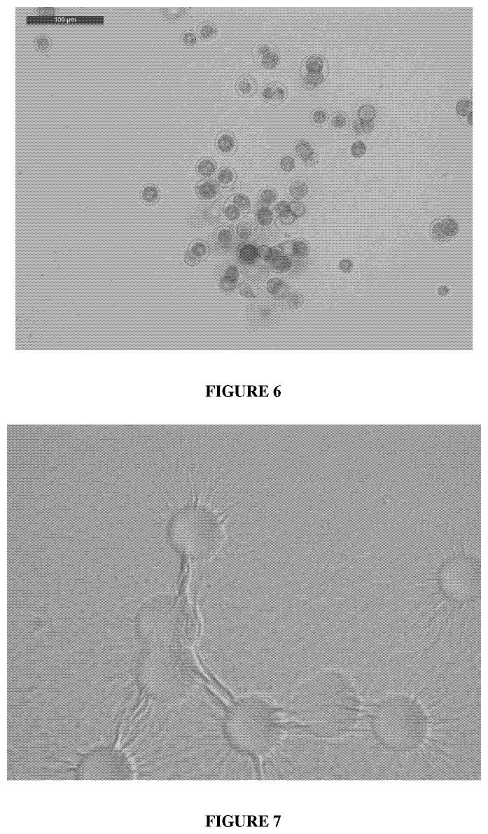

The response of the trophonts incubated in the autoclaved carrageenan buffer solutions at 26° C. is shown in FIG. 2. The efficiency of encystment in 0.125% carrageenan-H and 0.25% carrageenan-H was equivalent to the CI—H encystment. After 31 days, some of the encysted cells excysted into theront cells, however at a lesser rate carrageenan-H compared to CI—H. The cysts formed in carrageenan have distinctive capsules which are characteristic of typical T. rostrata cysts (see FIG. 6).

### Example 3: Carrageenan Buffer Solution Induces Encystment of T. rostrata at 20° C.

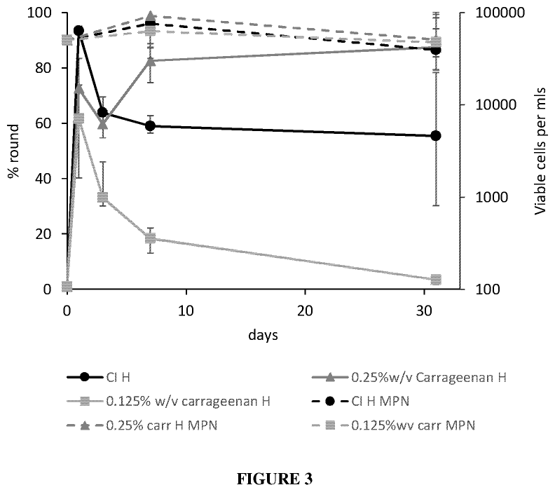

The response of the trophonts incubated in the autoclaved carrageenan buffer solutions at 20° C. is shown in FIG. 3. Encystment was tested carried out at 20° C. In CI—H there is a flux between encysted-excysted-re-encysted cells resulting in low overall cyst yield and a diversity in the cell population. This is seen in wide margins of error in cell counts. The number of cells that encysted in 0.25% carrageenan-H was greater than in 0.125% carrageenan-H or for the CI—H.

### Example 4: Encysted Cells in Carrageenan Buffer Solution Remain Viable and Encysted Following Transfer to 20° C.

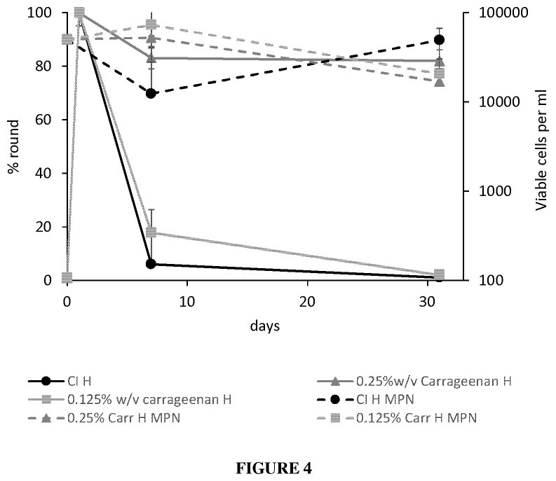

The response of the trophonts incubated in the autoclaved carrageenan buffer solutions at 26° C. followed by transfer to 20° C. is shown in FIG. 4. Cysts formed in CI—H at 26° C. excyst when they are transferred to 20° C. Cysts made in 0.25% carrageenan-H remained encysted at the lower temperature. This finding demonstrates that stable cysts can be made in a single step and may offer a simpler process than the two-step process with encystment followed by stabilisation with other additives (such as MgSO4, see below).

### Example 5: Stabilisation of Encysted Ciliate Cells with Magnesium Ions

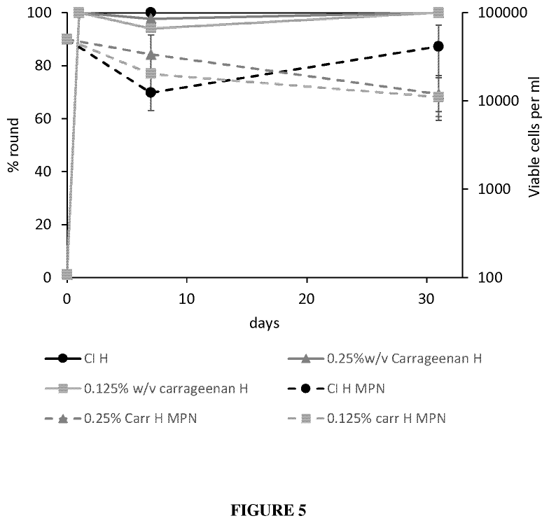

The effect of adding magnesium ions to the buffer solutions comprising encysted ciliate cells was evaluated and shown in FIG. 5. This treatment was tested by adding MgSO4 after 24 hours incubation at 26° C. but prior to transfer to 20° C. The MgSO4 prevented excystment of cells in CI—H and both carrageenan-H solutions, and did not affect viability of any of the cells in any treatment. The Addition of metal can be used to produce stable cysts with different concentrations of polymer.

### Example 6: Dehydrated Encysted Ciliate Cells in Carrageenan Remain Viable and Stable

The trophonts were suspended in 0.25% or 0.125% carrageenan-H and CI—H and were placed in humidity chambers at 20° C. suspended in humidity chambers above different saturated salts, sodium chloride or sodium bromide, which would equalise at relative humidity 75.7% or 59.1%, respectively. They were allowed to dehydrate at 20° C. The cell morphology in the dried carrageenan was assessed microscopically after 18 days and 31 days. All cells were round and cyst-like and were trapped in the dried film (FIG. 7).

The above examples show that T. rostrata TRAUS is capable of converting from trophonts to cysts in carrageenan solutions. Stable encystment in carrageenan is very efficient at both 20° C. and 26° C. and the cysts remained viable and stable.

### Example 7: Gellan Gum Encyst T. rostrata at High Efficiency

Carrageenan (Sigma C1013), LA-gellan gum (Kelcogel® F LA-gellan gum) and HA-gellan gum (Kelcogel® LT100 HA-gellan) were each dissolved separately in ultrapure water at room temperature to form a hydrocolloid suspension/solution. The carrageenan (CGN) was autoclaved at 121° C. for 20 minutes, and then cooled to room temperature. The gellan were not heat treated. The suspensions/solutions were then added at various concentrations to T. rostrata cells at 1-3×104 cell per ml and the culture was buffered with HEPES pH 7 (NaOH) at 10 mM to stimulate encystment at 26° C.

The encystment in carrageenan and gellan gum buffer solutions were compared to three control infusion buffer solutions prepared using compost pine bark (CI) and collected, dried and resuspended composted pine park particles (CDIP and DUP4) buffered with HEPES buffer pH 7 to a final concentration of 10 mM, and were then added at various concentrations to T. rostrata cells at 1-3×104 cell per ml and the culture was buffered with HEPES pH 7 (NaOH) at 10 mM to stimulate encystment at 26° C.

After 26 hours, the infusion buffer solutions were screened for their ability to encyst T. rostrata. Table 1 shows the proportion of T. rostrata cells that encysted using either the control pine bark infusion buffer solutions or the carrageenan/gellan gum buffer solution.

TABLE 1
The percent of T. rostrata cells stimulated to encyst by
extracts of composted pine bark (CI, DCIP, DUP4),
carrageenan (CGN) and gellan gum.

| | | | | | | LA | HA |
|---|---|---|---|---|---|---|---|
| | | | | | | gellan | gellan |
| | CI | DCIP | DUP4 | CGN | CGN # | gum | gum |
| | | | | Charge | | | |
| | | | | Anionic | Anionic | Anionic | Anionic |
| | Origin | | | | | | |
| | Plant | Plant | Plant | Seaweed | Seaweed | Bacteria | Bacteria |
| mg/mL | Percent of cells that encysted | | | | | | |
| 0.195 | 56 | 60 | 55 | 66 | 51 | 32 | 47 |
| 0.0381 | 77 | 59 | 79 | 65 | 52 | 38 | 66 |
| 0.0781 | 81 | 59 | 90 | 67 | 65 | 49 | 58 |
| 0.1563 | | 61 | 86 | 71 | 64 | 48 | 82 |
| 0.3125 | | 68 | 90 | 68 | 67 | 55 | 91 |
| 0.625 | | 82 | 92 | 81 | 83 | 43 | 91 |
| 1.25 | | 81 | 98 | 86 | 92 | 62 | |
| 2.5 | | 86 | 99 | | | 58 | |

¹CGN# was modified by the addition of HEPES pH7 (NaOH) at 10 mM prior to autoclaving.

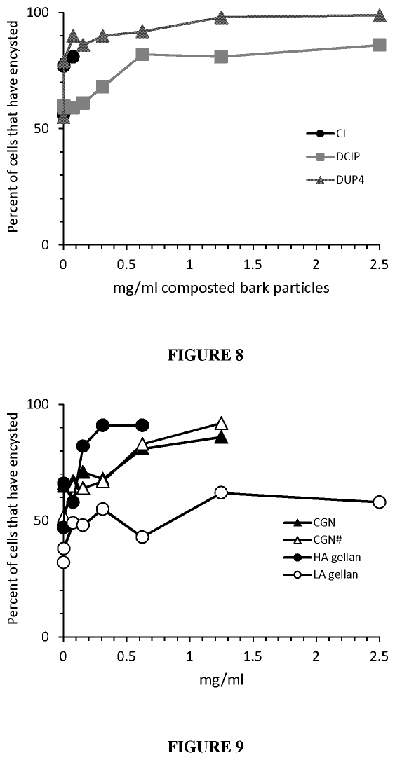

The encystment using the three control infusion buffers are shown in FIG. 8, highlighting the bark infusion preparations result in high efficiency of encystment. FIG. 9 shows the proportion of cells that encysted using preparations of carrageenan and gellan gum. Carrageenan and HA-gellan gum buffer solutions demonstrated high encystment efficiency T. rostrata.

### Example 8: Encystment of T. rostrata in Carrageenan Buffer Solutions Result in Durable Cysts

As carrageenan stimulates high efficiency of encystment of T. rostrata, the durability of the resulting cysts and infectivity of the excysted theronts was determined. The resulting cysts are durable and tolerated conditions like dehydration and certain salt concentrations. Carrageenan encysts T. rostrata when used in a buffer solution at 0.025% to 0.25% w/v. At the lower carrageenan concentration, 2 days is required for peak encystment, unlike 1 day at the higher carrageenan concentrations. Excystment of the theronts occurs at 20° C. when carrageenan is used in a buffer solution at 0.025% to 0.125% w/v. A higher concentration of carrageenan (0.25% w/v) resulted in a lower rate of excystment.

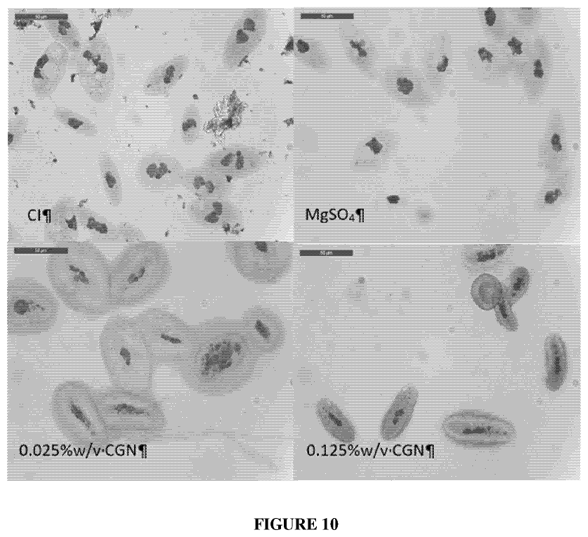

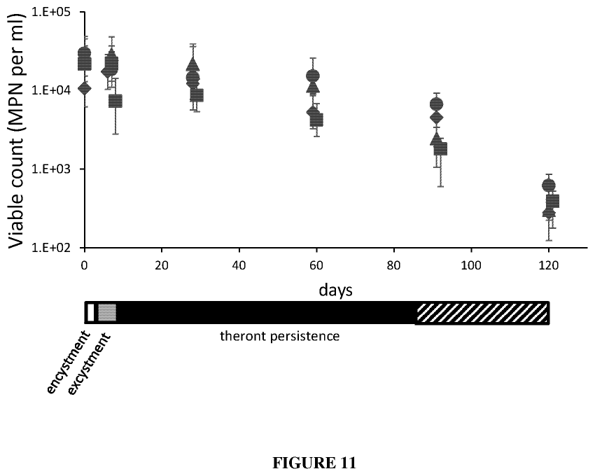

The theronts produced in carrageenan buffer solutions at 0.025% and 0.125% w/v show the same characteristic bi-lobed macronucleus and a micronucleus as those produced in buffered compost infusion (CI—H) or 62.5 μM MgSO4 when Giemsa stained (see FIG. 10).

Populations of theronts in the encystment buffer solutions at 20° C. were monitored for up to 120 days by determining viable counts, using Most Probable Number (MPN) assays (see FIG. 11). The MPN assay determines the number of viable cells present, which could multiply and establish a trophont culture. The culture was monitored by observing and counting cell morphology, as the cells changed from trophonts (day 0) through to cysts during starvation (day 1 to 3) and then theronts after excystment (day 5 to 7). The excysted cells were confirmed to be theronts by Giemsa staining and showed the characteristic bi-lobed macronucleus and a micronucleus.

Theronts produced and then stored at 20° C. in either buffered 0.025% w/v carrageenan persist for 2 months in a similar way to those in buffered CI (see FIG. 10). The theronts size remained stable. Theronts produced and then stored at 20° C. in either buffered 0.125% w/v carrageenan or 62.5 μM MgSO4 also persist for 2 months.

### Example 9: Encystment of T. rostrata in Carrageenan Buffer Solutions Result in Infective Theronts Following Excystment

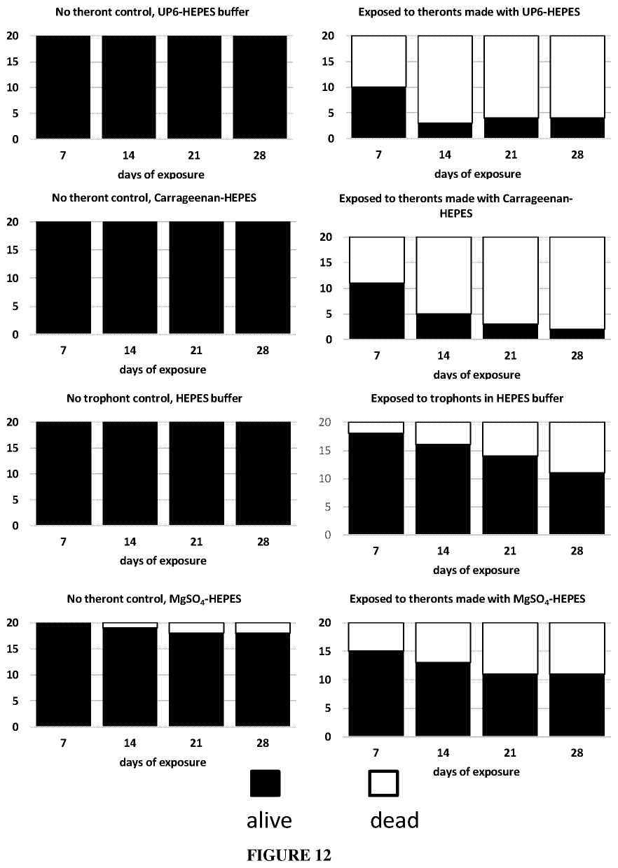

D. reticulatum lethality experiments were performed using theronts that had excysted from cysts produced in buffered 0.025% w/v carrageenan, and the results are shown in FIG. 12. D. reticulatum slugs were exposed to 1×105 theronts produced in buffered compost infusion (UP-6), 0.025% w/v carrageenan, or 62.5 μM MgSO4. They were also exposed to 1×105 trophonts in HEPES buffer. The survival of the slugs was monitored over 28 days.

The impact of the theronts made in compost infusion buffer (UP6) and carrageenan, on mortality and grazing was apparent after 7 days of exposure. And by 2 weeks 95% of the slugs exposed to theronts produced in compost infusion buffer (UP6) and carrageenan were affected and 16 or 15/20 of the slugs, respectively, were dead. In contrast, when slugs were exposed to theronts produced in 62.5 μM MgSO4 or trophonts more than ½ the slugs were alive at 28 days post exposure.

### Example 10: Encystment and Excystment of T. rostrata at Higher Cell Densities

Carrageenan (Sigma C1013) was dissolved in ultrapure water at room temperature to form a hydrocolloid suspension/solution, and autoclaved at 121° C. for 20 minutes, and then cooled to room temperature. 0.025% w/v carrageenan solution was then added at to T. rostrata cells at 3×105 cells per ml and the culture was buffered with HEPES pH 7 (NaOH) at 10 mM to stimulate encystment at 20° C. Control infusion buffer solutions were prepared using compost pine bark (CI) and collected, dried and resuspended composted pine park particles (UP6) buffered with HEPES buffer pH 7 to a final concentration of 10 mM, and then added to T. rostrata cells at 3×105 cell per ml and the culture was buffered with HEPES pH 7 (NaOH) at 10 mM to stimulate encystment at 20° C. After 26 hours, the infusion buffer solutions were screened for their ability to encyst and excyst T. rostrata at high cell densities (see Table 2).

TABLE 2
Theronts yield when T. rostrata is encysted &
excysted at 20° C. at a cell density of 3 × 10⁵ cell
per ml in various encystment buffers.

| | Culture at 8 days at 20° C. | | | |
|---|---|---|---|---|
| | (Cell density on day 1 was | | | |
| | 3 × 10⁵ trophonts per ml) | | | |
| | | | % of the | |
| | | | ciliated | |
| | | | cells | |
| Infusion | Cells | % | that are | Theronts |
| buffer | per ml | Ciliates | theronts | per ml |
| CI | 2.6 × 10⁵ | 86 | 73 | 1.6 × 10⁵ |
| UP6 | 3.5 × 10⁵ | 85 | 89 | 2.6 × 10⁵ |
| CGN | 2.0 × 10⁵ | 77 | 74 | 1.1 × 10⁵ |

### Example 11: Identification of Other Polysaccharides that Encyst T. rostrata

In addition to the polysaccharides carrageenan and gellan gum, a range of other polysaccharides were selected and sourced to screen for their ability to encyst T. rostrata, as outlined in Table 3. The polysaccharides were dissolved at a known concentration (either 0.125% w/v or 0.25% w/v) in stirred ultrapure water and heated to ensure full dissolution and hydration of the polysaccharide before cooling to room temperature to form an aqueous polysaccharide solution/suspension.

The cooled polysaccharide solutions/suspension was then added at 2-fold dilutions to T. rostrata at 1-3×104 cells per ml (low cell density). The cultures comprising the polysaccharide were then buffered with HEPES pH 7 (NaOH) at 10 mM and incubated at 26° C. for 26 hours. The percent of round/oval cells were counted, and these were determined to be cysts visually. For cysts a defined capsule layer around the protozoa with cells that are slightly compressed within the layer were seen. Many cells displayed cyclopsis (internal rotating movement) was occurring and the cells clustered and adhered together. To evaluate encystment efficiency, the encystment of T. rostrata cells in 10 mM HEPES pH 7 (NaOH) alone was used as a control, where 50% are cysts after 26 hours at 26° C. The type of polysaccharide and encystment results are shown in Table 3:

TABLE 3
The percent of T. rostrata cells stimulated to encyst by various polysaccharide buffer solutions at 26° C.
after 26 hours. Encystment of T. rostrata cells in 10 mM HEPES pH 7 (NaOH) alone was used as a control
where 50% are cysts after 26 hours at 26º C.

| | Polysaccharide | | | | | | | | | | | |
|---|---|---|---|---|---|---|---|---|---|---|---|---|
| | | | | | | | | | | Dextran | | |
| | | | | | | Fucoi- | Na- | | | sulphate | | |
| | k-CGN | | I-CGN | | λ-CGN | dian | alginate | Xanthan | Xanthan | 40 kDa | | CMC |
| | (Sigma | k-CGN | (Sigma | I-CGN | (Sigma | (Sigma | (Sigma | (MFD | (MFD | (Sigma | Guar | (Sigma |
| | C1013) | (MFD) | C1138) | (MFD) | 22049) | F8315) | A2033) | viscosity) | thickener) | 42867) | (MFD) | 419338) |
| | Charge | | | | | | | | | | | |
| | Anionic | Anionic | Anionic | Anionic | Anionic | Anionic | Anionic | Anionic | Anionic | Anionic | Neutral | Anionic |
| | Origin | | | | | | | | | | | |
| | Seaweed | Seaweed | Seaweed | Seaweed | Seaweed | Seaweed | Seaweed | Bacteria | Bacteria | Bacteria | Plant | Synthetic |
| | Heat treatment | | | | | | | | | | | |
| | 90- | 90- | 90- | 90- | 90- | 90- | 60°C | 90- | 90- | 17- | 17- | 17- |
| | 100° C. | 100° C. | 100° C. | 100° C. | 100° C. | 100° C. | | 100° C. | 100° C. | 21° C. | 21° C. | 21° C. |
| mg/mL | Percent of cells that encysted | | | | | | | | | | | |
| 0.00488 | 39 | | | | | | | | | | | |
| 0.00977 | 63 | | | | | | | | | | | |
| 0.01953 | 69 | | 51 | | | | | 52 | 58 | | | |
| 0.03906 | 77 | | 80 | | | | | 64 | 60 | | | 53 |
| 0.07813 | 84 | 52 | 84 | | | | | 83 | 87 | 56 | | 49 |
| 0.15625 | 95 | 53 | 88 | | 62 | 36 | 55 | 98 | 81 | 63* | 46 | 75* |
| 0.3125 | 93 | 79 | 95 | 51 | 68 | 51 | 40 | 98 | 99 | 76* | 53 | 74* |
| 0.625 | 97 | 96 | 100 | 70 | 73 | 33 | 67* | 100 | 99 | nd* | 58 | 71* |
| 1.25 | 98 | 98 | 97 | 79* | 84 | 75 | 66* | | | nd* | 64 | |
| 2.5 | | | | | | | | | | | | |

*Indicates presence of cell lysis.

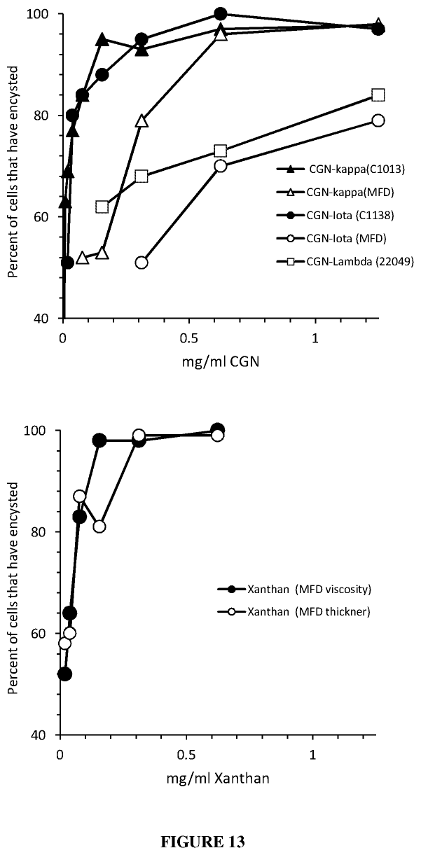

The encystment data demonstrates that a range of polysaccharides can encyst T. rostrata at higher percentages compared to the control using HEPES buffer alone as the encystment buffer (i.e. no polysaccharide). Consistent with the earlier examples, carrageenan buffer solutions encysted T. rostrata, with k-carrageenan (kappa) and I-carrageenan (iota) demonstrating high encystment efficiency (see FIG. 13). The anionic polysaccharide Xanthan gum encysted T. rostrata with very high efficiency (see FIG. 13).

### Example 12: Structural Form of the Polysaccharide May be Important to Encyst T. rostrata

The effect of heat treatment during hydrocolloid formation on T. rostrata encystment was investigated, as outlined in Table 4

The polysaccharides were dissolved at 0.125% w/v in stirred heated to 90-100° C. or 50-60° C. ultrapure water to ensure full dissolution and hydration of the polysaccharide before cooling to room temperature or in stirred ultrapure water at room temperature (17-20° C.). The polysaccharide solutions/suspension was then added at 2-fold dilutions to T. rostrata at 1-3×104 cells per ml (low cell density). The cultures comprising the polysaccharide were then buffered with HEPES pH 7 (NaOH) at 10 mM and incubated at 26° C. for 26 hours. The percent of round/oval cells were counted, and these were determined to be cysts visually. For cysts a defined capsule layer around the protozoa with cells that are slightly compressed within the layer were seen. For many slight cyclopsis (internal rotating movement) was occurring and the cells clustered and adhered together.

To evaluate encystment efficiency, the encystment of T. rostrata cells in 10 mM HEPES pH 7 (NaOH) alone was used as a control, where 50% are cysts after 26 hours at 26° C. The type of polysaccharide and encystment results are shown in Table 4:

TABLE 4
The percent of T. rostrata cells stimulated to encyst by various
polysaccharide buffer solutions at 26° C. after 26 hours. Encystment
of T. rostrata cells in 10 mM HEPES pH 7 (NaOH) alone was
used as a control where 50% are cysts after 26 hours at 26° C.

| | Polysaccharide | | | |
|---|---|---|---|---|
| | k-CGN | HA-gellan | HA-gellan | HA-gellan |
| | (90-100° C.) | (90-100° C.) | (50-60° C.) | (17-20° C.) |
| | Charge | | | |
| | Anionic | Anionic | Anionic | Anionic |
| | Origin | | | |
| | Seaweed | Bacteria | Bacteria | Bacteria |
| mg/mL | Percent of cells that encysted | | | |
| 0.01953 | 62 | | | |
| 0.03906 | 81 | | | 54 |
| 0.07813 | 71 | | | 63 |
| 0.15625 | 96 | 54 | 48 | 91 |
| 0.3125 | 88 | 49 | 58 | 96 |
| 0.625 | 91 | 67 | 62 | 96 |
| 1.25 | 99 | | | |

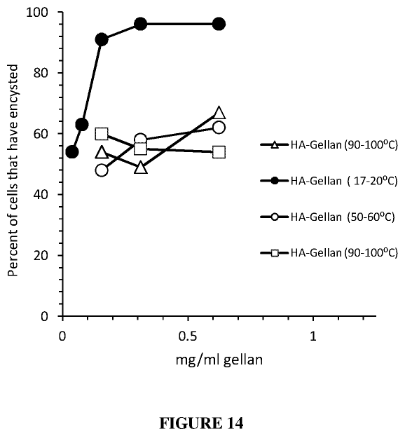

Heating and cooling the HA-gellan likely changes the structural matrix that this hydrocolloid forms. The encystment data suggests that the structure matrix of polysaccharides is important to encyst T. rostrata at higher percentages compared to the control using HEPES buffer alone as the encystment buffer (i.e. no polysaccharide). HA gellan that has not been heat-cooled encysted T. rostrata with high encystment efficiency (see FIG. 14).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

The present application claims priority from AU2021900775 filed on 17 Mar. 2021, the entire contents of which are incorporated herein by reference.

### REFERENCES

Billman-Jacobe et al. (2020), Biocontrol Sci. Technol 30: 920-928
Parhi et al. (2017) Adv Pharm Bull 7:515-530
Segade et al. (2016) Parasitol Res 115:771-777

1. A composition comprising encysted ciliate cells that are interspersed within a non-ionically crosslinked polymer.

2. The composition of claim 1, wherein the polymer comprises a polysaccharide.

3. The composition of claim 2, wherein the polysaccharide is an anionic polysaccharide.

4. The composition of claim 2 or claim 3, wherein the polysaccharide is derived from one or more of algae, plants, animals, fungi, or bacteria.

5. The composition of any one of claims 2 to 4, wherein the polysaccharide is an algal polysaccharide or a bacterial polysaccharide.

6. The composition of claim 5, wherein the polysaccharide is selected from the group consisting of carrageenan, agarose, agar, alginate, xanthan gum and gellan gum, or a mixture thereof.

7. The composition of any one of claims 1 to 6, wherein the polysaccharide is carrageenan, xanthan gum or gellan gum, or a mixture thereof.

8. The composition of any one of claims 1 to 7, wherein the polymer is dispersed in an aqueous solution, and the encysted ciliate cells are suspended in the aqueous solution comprising the polymer.

9. The composition of claim 8, wherein the aqueous solution comprising the polymer forms a hydrocolloid.

10. The composition of any one of claims 1 to 9, wherein the aqueous solution comprises between about 0.01% w/v to about 5% w/v polymer based on the total volume of the aqueous solution.

11. The composition of any one of claims 1 to 10, wherein the aqueous solution comprises between about 0.01% w/v to about 1% w/v polymer based on the total volume of the aqueous solution.

12. The composition of any one of claims 1 to 11, wherein the aqueous solution is a buffer solution.

13. The composition of claim 12, wherein the buffer solution comprises a HEPES buffering agent.

14. The composition of claim 13, wherein the concentration of the HEPES buffering agent in the buffer solution is between about 5 mM to about 25 mM.

15. The composition of any one of claims 12 to 14, wherein the buffer solution has a pH of between about 6.0 to about 9.0.

16. The composition of any one of claims 1 to 15, wherein the encysted ciliate cells interspersed within the polymer remain viable for at least about four weeks.

17. The composition of any one of claims 1 to 16, wherein the ciliate cells are a member of the Ciliophora phylum.

18. The composition of any one of claims 1 to 17, wherein the ciliate cells are a member of the Heterotrichea, Karyorelictea, Armophorea, Litostomatea, Colpodea, Nassophorea, Phyllopharyngea, Prostomatea, Plagiopylea, Oligohymenophorea, Protocruziea, Spirotrichea, or Cariotrichea class.

19. The composition of any one of claims 1 to 18, wherein the ciliate cells are a member of the Apostomatia, Astomatia, Hymenostomatia, Peniculia, Peritrichia, or Scuticociliatia order.

20. The composition of any one of claims 1 to 19, wherein the ciliate cells are a member of the Tetrahymenidae, Ophryoglenina, or Peniculina family.

21. The composition of any one of claims 1 to 20, wherein the ciliate cells are a member of the Tetrahymena genus.

22. The composition of any one of claims 1 to 21, wherein the ciliate cells are of the T. rostrata, T. hegewischi, T. hyperangularis, T. malaccensis, T. patula, T. pigmentosa, T. foissneri, T. deweyae, T. pyriformis, T. thermophila, T. vorax, T. geleii, T. corlissi, T. empidokyrea or T. limacis species.

23. The composition of any one of claims 1 to 22, wherein the ciliate cells are of the T. rostrata species.

24. A method of inducing the encystment of ciliate cells, the method comprising incubating a population of trophont ciliate cells in an aqueous solution comprising a polymer at a temperature and for a period of time effective to induce the encystment of the trophont ciliate cells to form one or more encysted ciliate cells.

25. A method of stabilising encysted ciliate cells, the method comprising suspending a population of encysted ciliate cells in an aqueous solution comprising a polymer.

26. The method of claim 24 or claim 25, wherein the aqueous solution comprising the polymer forms a hydrocolloid.

27. The method of any one of claim 25 to claim 26, wherein the polymer is not ionically crosslinked during encystment.

28. The method of any one of claims 24 to 27, wherein the polymer comprises a polysaccharide.

29. The method of claim 28, wherein the polysaccharide is an anionic polysaccharide.

30. The method of claim 28 or claim 29, wherein the polysaccharide is derived from one or more of algae, plants, animals, fungi, or bacteria.

31. The method of any one of claims 28 to 30, wherein the polysaccharide is derived from algae or bacteria.

32. The method of claim 31, wherein the polysaccharide is selected from the group consisting of carrageenan, agarose, agar, alginate, xanthan gum and gellan gum, or a mixture thereof.

33. The method of any one of claims 24 to 32, wherein the polysaccharide is carrageenan, xanthan gum or gellan gum, or a mixture thereof.

34. The method of any one of claims 24 to 33, wherein the aqueous solution comprises between about 0.01% w/v to about 5% w/v polymer based on the total volume of the aqueous solution.

35. The method of any one of claims 24 to 34, wherein the aqueous solution comprises between about 0.01% w/v to about 1% w/v polymer based on the total volume of the aqueous solution.

36. The method of claim 24 or any one of claims 25 to 35, wherein the trophont ciliate cells are incubated in the aqueous solution at a temperature of between about 10° C. to about 30° C.

37. The method of claim 24, or any one of claims 25 to 36, wherein the trophont ciliate cells are incubated in the aqueous solution for period of time of between about 12 to about 72 hours.

38. The method of any one of claims 24 to 37, wherein the aqueous solution is a buffer solution.

39. The method of claim 38, wherein the buffer solution comprises a HEPES buffering agent.

40. The method of claim 39, wherein the concentration of the HEPES buffering agent in the buffer solution is between about 1 mM to about 25 mM.

41. The method of any one of claims 38 to 40, wherein the buffer solution has a pH of between about 6.0 to about 9.0.

42. The method of any one of claims 24 to 41, further comprising the step of adding magnesium ions to the aqueous solution prior to or during incubation in an amount effective to stimulate encystment of one or more trophont ciliate cells.

43. The method of any one of claims 24 to 42, further comprising the step of adding magnesium ions to the aqueous solution following incubation in an amount effective to stabilise the one or more encysted ciliate cells.

44. The method of claim 43, wherein the magnesium ions are provided by magnesium sulfate.

45. The method of any one of claims 24 to 34, wherein the encysted ciliate cells are stored in the aqueous solution at a temperature of between about 10° C. to about 30° C.

46. The method of any one of claims 24 to 45, further comprising dehydrating the aqueous solution to obtain a dried polymer wherein the one or more encysted ciliate cells are interspersed within the polymer.

47. The method of any one of claims 24 to 46, wherein the ciliate cells are a member of the Ciliophora phylum.

48. The method of any one of claims 24 to 47, wherein the ciliate cells are a member of the Heterotrichea, Karyorelictea, Armophorea, Litostomatea, Colpodea, Nassophorea, Phyllopharyngea, Prostomatea, Plagiopylea, Oligohymenophorea, Protocruziea, Spirotrichea, or Cariotrichea class.

49. The method of any one of claims 24 to 48, wherein the ciliate cells are a member of the Apostomatia, Astomatia, Hymenostomatia, Peniculia, Peritrichia, or Scuticociliatia order.

50. The method of any one of claims 24 to 49, wherein the ciliate cells are a member of the Tetrahymenidae, Ophryoglenina, or Peniculina family.

51. The method of any one of claims 24 to 50, wherein the ciliate cells are a member of the Tetrahymena genus.

52. The method of any one of claims 24 to 51, wherein the ciliate cells are of the T. rostrata, T. hegewischi, T. hyperangularis, T. malaccensis, T. patula, T. pigmentosa, T. foissneri, T. deweyae, T. pyriformis, T. thermophila, T. vorax, T. geleii, T. corlissi, T. empidokyrea or T. limacis species.

53. The method of any one of claims 24 to 52, wherein the ciliate cells are of the T. rostrata species.

54. An encystment media composition comprising an aqueous solution and a non-ionically crosslinked polymer.

55. The composition of claim 54, wherein the aqueous solution comprising the polymer forms a hydrocolloid.

56. The composition of claim 54 or claim 55, wherein the polymer comprises a polysaccharide.

57. The composition of claim 56, wherein the polysaccharide is an anionic polysaccharide.

58. The composition of claim 56 or claim 57, wherein the polysaccharide is derived from one or more of algae, plants, animals, fungi, or bacteria.

59. The composition of any one of claims 56 to 58, wherein the polysaccharide is derived from algae.

60. The composition of claim 59, wherein the algal polysaccharide is selected from the group consisting of carrageenan, agarose, agar and alginate, or a mixture or copolymer thereof.

61. The composition of any one of claims 54 to 60, wherein the polysaccharide is carrageenan.

62. The composition of any one of claims 54 to 61, wherein the aqueous solution comprises between about 0.1% w/v to about 5% w/v polymer based on the total volume of the aqueous solution.

63. The composition of any one of claims 54 to 62, wherein the aqueous solution comprises between about 0.1% w/v to about 1% w/v polymer based on the total volume of the aqueous solution.

64. The composition of any one of claims 54 to 63, wherein the aqueous solution is a buffer solution.

65. The composition of claim 64, wherein the buffer solution comprises a HEPES buffering agent.

66. The composition of claim 65, wherein the concentration of the HEPES buffering agent in the buffer solution is between about 1 mM to about 25 mM.

67. The composition of any one of claims 64 to 66, wherein the buffer solution has a pH of between about 6.0 to about 9.0.

68. An agricultural or horticultural composition comprising the composition of any one of claims 1 to 23 or an encysted ciliate cell prepared or stabilised by the method according to any one of claims 24 to 53, in combination with one or more agriculturally or horticulturally acceptable carriers.

69. A method of infecting or colonising a pest species with a ciliate, the method comprising applying to an area affected or likely to be affected by a pest species a composition of any one of claim 1 to 23 or 68 or an encysted ciliate cell prepared or stabilised by the method according to any one of claims 24 to 53.

70. A method of infecting or colonising a pest species with a ciliate, the method comprising applying a composition of any one of claim 1 to 23 or 68 or an encysted ciliate cell prepared or stabilised by the method according to any one of claims 24 to 53 to the pest species

71. A method for reducing agricultural crop damage comprising applying to an agricultural crop area affected or likely to be affected by a pest species a pest species a composition of any one of claim 1 to 23 or 68 or an encysted ciliate cell prepared or stabilised by the method according to any one of claims 24 to 53.

72. The method of any one of claims 69 to 71, which results in the ciliate killing or affecting the fitness of the pest species.

73. The method of any one of claims 69 to 72, wherein the pest species is an invertebrate.

74. The method of claim 73, wherein the pest species is a mollusc.

75. The method of claim 74, wherein the mollusc is a Gastropod.

76. The method of claim 75, wherein the Gastropod is a snail or slug.
